(19) United States
(12) Patent Application Publication
(10) Pub. No.: US 2003/0219785 A1
(43) Pub. Date: Nov. 27, 2003

(54) Targeted drug delivery methods

(75) Inventors: Dennis E. Hallahan, Nashville, TN (US); Ling Geng, Nashville, TN (US); Todd D. Giorgio, Nashville, TN (US)

Correspondence Address: JENKINS & WILSON, PA, 3100 TOWER BLVD, SUITE 1400, DURHAM, NC, 27707, US

(73) Assignee: Vanderbilt University

(21) Appl. No.: 10/355,824

(22) Filed: Jan. 31, 2003

Related U.S. Application Data
(60) Provisional application No. 60/353,306, filed on Feb. 1, 2002.

(51) Int. Cl. C12Q 1/68

(52) U.S. Cl. 435/6

(57) ABSTRACT

The identification of radiation-inducible genes by isolating RNA from irradiated cell cultures and then hybridizing the isolated RNA to nucleic acid sequences from an organism of interest (e.g. mammals such as mice and human beings); a method for x-ray guided drug delivery using a targeting ligand that specifically recognizes a radiation-inducible RNA target molecule; and magnetic dispersion of an active agent, such as the dispersion of a genetic construct within a tumor.

### CROSS REFERENCE TO RELATED APPLICATIONS

The present patent application is based on and claims priority to U.S. Provisional Application Serial No. 60/353,306, entitled “TARGETED DRUG DELIVERY METHODS”, which was filed Feb. 1, 2002 and is incorporated herein by reference.

### GRANT STATEMENT

This invention was made in part from government support under Grant Nos. CA70937 and CA58508 from the National Institute of Health. Thus, the U.S. Government has certain rights in the invention.

### FIELD OF THE INVENTION

The present invention relates, in general, to targeted drug delivery methods. More particularly, the present invention relates to the identification and targeting of radiation inducible gene transcripts and to the use of magnetically targetable delivery vehicles to enhance biodistribution of an active agent, such as a genetic construct.

Table of Abbreviations

| | |
|---|---|
| Ad | Adenovirus or adenoviral |
| Ad. LacZ | adenoviral-beta-galactosidase |
| | expression vector |
| AVM | arteriovenous malformation(s) |
| BPR | bovine pancreatic ribonuclease |
| BSA | bovine serum albumin |
| C57BL6J | strain of mice |
| CAM | cell adhesion molecule |
| CaMV | Cauliflower mosaic virus |
| CEA | carcinoembryonic antigen |
| cGy | centiGray |
| CT | computed tomography |
| DAPI | 4',6-diamindino-2-phenylindole |
| | dihydrochloride |
| DMF | dimethylformamide |
| DMSA | dimercaptosuccinic acid |
| DNA | deoxyribonucleic acid |
| DOPE | dioleyol phosphatidyl ethanolamine |
| dsRNAs | double-stranded RNAs |
| DT | diphtheria toxin |
| DTPA | diethylenetriamine pentaacetate |
| EDC | 1-ethyl-3 [3- (dimethylamino) propyl] |
| | carbodiimide |
| EDTA | ethylenediaminetetraacetic acid |
| ELISA | enzyme linked immunosorbent assay |
| FITC | fluorescein isothiocyanate |
| GEL | gelonin |
| GI-261 | tumor model |
| GM-CSF | granulocyte-macrophage colony- |
| | Stimulating factor |
| GP-IIb | platelet membrane glycoprotein IIb |
| GP-IIIa | platelet membrane glycoprotein IIIa |
| GST | glutathione S-transferase |
| Gy | Gray |
| h or hr | hour(s) |
| H460 | tumor model |
| HEDTA | N-(2-hydroxyethyl) |
| | ethylenediaminetriacetic acid |
| Her2/neu | v-erb-b2 avian erythroblastic leukemia |
| | viral oncogene homologue 2 |
| HLA | human leukocyte antigen |
| HRP | horseradish peroxidase |
| HSA | horse serum albumin |
| Ig | immunoglobulin |
| lgG | immunoglobulin G |
| IL-12 | interleukin 12 |
| IL-2 | interleukin 2 |
| IL-4 | interleukin 4 |
| IL-7 | interleukin 7 |
| ITRs | Invented Terminal Repeats |
| IV | intravenously (IV) |
| keV | kiloelectron volts |
| kV | kilovolt(s) |
| LLC | tumor model |
| LTRs | Long Terminal Repeats |
| M | molar |
| mCi | millicurie |
| mg | milligram(s) |
| min | minute(s) |
| ml | milliliter(s) |
| mm | millimolar |
| MRI | nuclear magnetic resonance imaging |
| MV | megavolt(s) |
| nM | nanomoles or nanomolar |
| NTA | nitrilotriacetic acid |
| PAP | pokeweed antiviral protein |
| PBS | phosphate buffered saline |
| PCR | polymerase chain reaction |
| PE | Pseudomonas exotoxin |
| PEG | polyethylene glycol |
| pfu | plaque forming units |
| PK | protein kinase |
| Pt | platinum |
| RES | reticular endothelial system |
| RNA | ribonucleic acid |
| RNAi | RNA interference |
| RSVE | reconstituted Sendai virus envelopes |
| RT | room temperature |
| SAP | saporin |
| SD | standard deviation |
| SMPT | 4-succinimidyloxycarbonyl-methyl- |
| | (2-pyridyldithio)-toluene |
| SPDP | N-succinimidyl-3-(2-pyridyldithio) |
| | propionate |
| SPECT | single photon emission computed |
| | tomography |
| Tie-2 | endothelium-specific receptor tyrosine |
| | kinase |
| TMV | Tobacco mosaic virus |
| TNF-α | tumor necrosis factor alpha |
| VEGF | vascular endothelial growth factor |
| WGA | wheat germ agglutinin |
| WPB | Weibel-Palade body |
| μg | microgram(s) |
| μl | microliter(s) |

### BACKGROUND ART

Currently practiced methods of tissue specific drug delivery, including tumor specific drug delivery, involve the use of antibody conjugates to liposomes and viral vectors. For tumors, these methods are specific for tumor subtype or are nonspecific in localization. These limitations are significant in that, on the one hand, only certain types of tumors may be treated and, on the other hand, nonspecific localization produces undesirable collateral damage to otherwise healthy tissue.

Approaches for the dispersion of gene therapy vectors is another ongoing and long-felt need in the art. Viral vectors and other gene therapy vectors administered to cancer patients often have suboptimal biodistribution. In some cases, intravenously (IV)-administered vectors distribute nonspecifically throughout the entire circulation. For example, adenovirus-based vectors can bind within the liver following IV administration. Poor biodistribution has also been seen when intratumoral injection of gene therapy is utilized. In some cases, therapeutic gene expression has been observed to be limited to needle tracks. Homogenous gene expression also occurs following distribution of inhalation vectors.

Thus, there remains significant need in the field for advances in the tissue-selective delivery and enhanced biodistribution of therapeutic and imaging agents. Moreover, there remains a substantial need in the art for an improved method and composition for the selective delivery of therapeutic or imaging agents to neoplastic tissue, as well as and enhanced biodistribution within neoplastic tissue. The present invention addresses these and other needs in the art.

### SUMMARY OF THE INVENTION

A method for identifying a radiation-inducible gene is disclosed. In one embodiment the method comprises: (a) isolating RNA from an irradiated cell; (b) hybridizing the isolated RNA to one or more nucleic acids from a subject; and (c) detecting hybridization between the isolated RNA and the one or more nucleic acids to thereby identify a radiation-inducible gene.

Optionally, the irradiated cell is a cell from a cell culture or from a tissue sample. The tissue sample can be derived from a warm-blooded vertebrate, such as a human.

The isolated RNA can comprise a detectable label. The one or more nucleic acids can be selected from the group consisting of a deoxyribonucleic acid, a ribonucleic acid, and a combination thereof. Also, the one or more nucleic acids each comprise a nucleotide sequence encoding a polypeptide. Additionally, at least one of the one or more nucleic acids can comprise a detectable label.

The one or more nucleic acids can be immobilized on a solid substrate comprising a plurality of identifying positions, each of the one or more nucleic acids occupying one of the plurality of identifying positions. The solid substrate can comprise silicon, glass, plastic, polyacrylamide, a polymer matrix, an agarose gel, a polyacrylamide gel, an organic membrane, or an inorganic membrane.

A method of delivering an active agent to a target tissue in a vertebrate subject is also disclosed. In one embodiment the method comprises: (a) providing a delivery vehicle comprising an active agent and a targeting agent that binds a radiation-induced RNA molecule; (b) exposing the target tissue to ionizing radiation; and (c) administering a delivery vehicle to the vertebrate subject before, after, during, or combinations thereof, exposing the target tissue to the ionizing radiation, whereby the delivery vehicle localizes to a radiation-induced RNA molecule in the target tissue to thereby deliver the active agent to the target tissue.

A delivery vehicle for use in targeted delivery of an active agent is also disclosed. In one embodiment, the delivery vehicle comprises a targeting agent that binds a radiation inducible RNA molecule in a target tissue.

A method of enhancing retention of an active agent in a target tissue in a vertebrate subject is also disclosed. In one embodiment, the method comprises: (a) providing a delivery vehicle comprising an active agent, a paramagnetic material, and a targeting agent that binds a radiation-induced target molecule; (b) exposing the target tissue to ionizing radiation; (c) exposing the target tissue to a magnetic field; and (d) administering a delivery vehicle to the vertebrate subject, whereby the delivery vehicle localizes to and is retained in the target tissue.

A method of dispersing a genetic construct in a target tissue is also disclosed. In one embodiment, the method comprises: (a) providing a delivery vehicle comprising a genetic construct and a paramagnetic material; (b) administering the delivery vehicle to a target tissue; and (c) applying a magnetic field to the target tissue to thereby disperse the genetic construct.

In any of the foregoing embodiments, the targeting agent can be selected from the group consisting of an antibody and a nucleic acid. Optionally, the nucleic acid is a double-stranded RNA.

In any of the foregoing embodiments, the active agent can comprise an imaging agent, such as a paramagnetic, radioactive and/or fluorogenic ions. The radioactive imaging agent can be selected from the group consisting of gamma-emitters, positron-emitters and x-ray-emitters. Also, the radioactive imaging agent can be selected from the group consisting of ⁴³K, ⁵²Fe, ⁵⁷Co, ⁶⁷Cu, ⁶⁷Ga, ⁶⁸Ga, ⁷⁷Br, ⁸¹Rb/^{81M}Kr, ^{87M}Sr, ^{99M}Tc, ¹¹¹In, ¹¹³In ¹²³I, ¹²⁵I, ¹²⁷Cs, ¹²⁹Cs, ¹³¹I, ¹³²I, ¹⁹⁷Hg, ²⁰³Pb and ²⁰⁶Bi. The radioactive imaging agent can be present in an amount ranging from about 0.1 to about 100 millicuries.

In any of the foregoing embodiments, the active agent can comprise a therapeutic agent. The therapeutic agent can be selected from the group consisting of a chemotherapeutic agent, a toxin, a radiotherapeutic agent, a radiosensitizing agent, a genetic construct, and combinations thereof. The chemotherapeutic agent can be selected from the group consisting of an anti-tumor drug, a cytokine, an anti-metabolite, an alkylating agent, a hormone, methotrexate, doxorubicin, daunorubicin, cytosine arabinoside, etoposide, 5-4 fluorouracil, melphalan, chlorambucil, a nitrogen mustard, cyclophosphamide, cis-platinum, vindesine, vinca alkaloids, mitomycin, bleomycin, purothionin, macromomycin, 1,4-benzoquinone derivatives, trenimon, steroids, aminopterin, anthracyclines, demecolcine, etoposide, mithramycin, doxorubicin, daunomycin, vinblastine, neocarzinostatin, macromycin, -amanitin, and combinations thereof.

The toxin can be selected from the group consisting of Russell's Viper Venom, activated Factor IX, activated Factor X, thrombin, phospholipase C, cobra venom factor, ricin, ricin A chain, Pseudomonas exotoxin, diphtheria toxin, bovine pancreatic ribonuclease, pokeweed antiviral protein, abrin, abrin A chain, gelonin, saporin, modeccin, viscumin, volkensin and combinations thereof.

The radiotherapeutic agent can be selected from the group consisting of ⁴⁷Sc, ⁶⁷Cu, ⁹⁰Y, ¹⁰⁹Pd, ¹²³I, ¹²⁵I, ¹³¹I, ¹⁸⁶Re, ¹⁸⁸Re, ¹⁹⁹Au, ²¹¹At, ²¹²Pb, ²¹²Bi, ³²P, ³³P, ⁷¹Ge, ⁷⁷As, ¹⁰³Pb, ¹⁰⁵Rh, ¹¹¹Ag, ¹¹⁹Sb, ¹²¹Sn, ¹³¹Cs, ¹⁴³Pr, ¹⁶¹Tb, ¹⁷⁷Lu, ¹⁹¹Os, 193MPt, and ¹⁹⁷Hg.

The radiosensitizing agent can be selected from the group consisting of an anti-angiogenic agent; a DNA protein kinase inhibitor; a tyrosine kinase inhibitor; a DNA repair enzyme inhibitor; nitroimidazole; metronidazole; misonidazole; a genetic construct comprising an enhancer-promoter region which is responsive to radiation, and at least one structural gene whose expression is controlled by the enhancer-promoter; boron-neutron capture reagents; and combinations thereof. The genetic construct further can comprises a viral vector.

Optionally, the therapeutic agent is a chemotherapeutic agent, and the delivery vehicle comprising the chemotherapeutic agent is administered in an amount ranging from about 10 mg to about 1000 mg. Optionally, the therapeutic agent is a toxin, and the delivery vehicle comprising the toxin is administered in an amount ranging from about 1 to about 500 μg. Optionally, the therapeutic agent is a radiotherapeutic agent, and the delivery vehicle comprising the radiotherapeutic agent is administered in an amount ranging from about 0.5 mg to about 100 mg.

The target tissue can comprise a neoplasm. The vertebrate subject can comprise a mammal, such as a human.

In any of the foregoing embodiments, the paramagnetic material is selected from the group consisting of iron and gadolinium, the paramagnetic material further comprising a material that exhibits a photoelectric effect upon interaction with incident radiation, and/or the paramagnetic material is in the form of a nanoparticle.

In any of the foregoing embodiments, the delivery vehicle can comprises a linker that links the paramagnetic material and the genetic construct. Optionally, the linker is a peptide. Also optionally, the linker is a cleavable linker.

Thus, it is an object of the present invention to provide a novel method and composition for targeted delivery of an active agent.

An object of the invention having been stated hereinabove, and which is addressed in whole or in part by the present invention, other objects will become evident as the description proceeds when taken in connection with the accompanying drawings as best described hereinbelow.

### BRIEF DESCRIPTION OF THE DRAWINGS

FIG. 1 is a schematic of a delivery vehicle as disclosed herein, wherein the delivery vehicle comprises a magnetic nanoparticle and a genetic construct, and the magnetic nanoparticle and the genetic construct are linked via an avidin/streptavidin/biotin linker.

FIGS. 2A-2C are photographs depicting magnetic dispersion of intratumoral vectors.

FIGS. 3A and 3B are a schematic of a delivery vehicle of the present invention wherein the delivery vehicle comprises a magnetic nanoparticle and a genetic construct, and the magnetic nanoparticle and genetic construct are linked by protein-antibody linkers.

FIGS. 4A and 4B are a schematic of delivery vehicle as disclosed herein, wherein the delivery vehicle comprises a magnetic nanoparticle and a genetic construct, and the magnetic nanoparticle the genetic construct are linked by a linker comprising a chelated metal ion and polyhistidine interaction. FIG. 4B is an expanded view of a fiber structure in the genetic construct.

FIGS. 5A and 5B are a schematic view of a delivery vehicle as disclosed herein, wherein the delivery vehicle comprises a magnetic nanoparticle and a genetic construct, and the genetic construct and magnetic particle are linked by a linker comprising a protein and polylysine. FIG. 5B is an expanded view of a fiber structure of the genetic construct.

FIG. 6 is a schematic of a delivery vehicle of the present invention wherein the delivery vehicle comprises a magnetic nanoparticle and a genetic construct, and the magnetic nanoparticle and genetic construct are linked via an interaction between polyethylene glycol and a liposome.

FIGS. 7A and 7B show LLC mouse tumor models that were irradiated with 0 Gy (FIG. 7A) and (FIG. 7B) Thirty (30) minutes after irradiation, WGA-biotin was injected into the blood flow via the tail vein, and 10 μm frozen sections were cut from the tumor excised at 30 minutes after injection. Avidin-FITC was used to stain for WGA, and DAPI was used for counterstaining. Green fluorescence shows the binding of WGA to the irradiated endothelial cells. The sections were taken from the peripheral area of the tumor, which has the greatest supply of the blood vessels.

FIGS. 8A and 8B are photographs of LLC window models, which were irradiated with 2.5 Gy (FIG. 8A) and 0 Gy (FIG. 8B). One hour after the irradiation, 100 μl of WGA labeled with FITC was injected into the blood flow via the tail vein. FIGS. 8A and 8B were taken one hour after that. On FIG. 8A, the green fluorescent spots along the blood vessels (bright spots on dark vessels) show that WGA has a much greater binding ability to the inflamed irradiated vasculature than to the one without irradiation (FIG. 8B).

FIGS. 9A and 9B are x-ray photographs of LLC bearing mouse. FIG. 9A shows the tumor on the right leg without vasculature image. FIG. 9B shows the same mouse injected with 300 μl of paramagnetic-DPTA delivery vehicle and exposed to magnet for 15 minutes. In FIG. 9B, the arrows point to two blood vessels.

FIGS. 10A-10D are photographs of LLC and GL-261 tumor models, which were used to test WGA-paramagnetic delivery vehicle pulled to tumor by magnet. WGA as the marker was stained with anti-WGA antibody, alkaline phosphatase and substrate kit for WGA (dark area), Eosin staining as a counter stain (lighter areas). FIG. 10A, LLC, WGA-paramagnetic delivery vehicle without magnet; FIG. 10B, LLC, WGA-paramagnetic delivery vehicle with magnet; FIG. 10C, GL-261, WGA-paramagnetic delivery vehicle without magnet; FIG. 10D, GL-261, WGA-paramagnetic delivery vehicle with magnet.

FIGS. 11A-11D are tumor volume change curves from GI-261 mouse tumor models, which were used to test the delivery vehicles. FIG. 11A presents a summary curve, while FIGS. 11B-11D are broken down into different treatment groups. The following symbols are employed: diamond, control; square, irradiation (3 Gy) 2X; triangle, cisplatin (0.08 mg/100 ml), 4X; plus sign, irradiation+paramagnetic+WGA+cisplatin+magnet; minus sign, irradiation+paramagnetic+cisplatin+magnet; asterisk, irradiation+param agnetic+WGA+cisplatin; X, paramagnetic+WGA+cisplatin; solid circle, irradiation+cisplatin.

FIG. 12 is a H460 tumor volume change curve with the treatments of irradiation, cisplatin, and combinations. The following symbols are employed: diamond, control; square, irradiation (3 Gy) 2X; triangle, irradiation+cisplatin (0.08 mg/100 ml), 4X; X, irradiation+paramagnetic+WGA+cisplatin+magnet;

FIGS. 13A and 13B are histograms depicting Doppler data indicating blood flow in the peripheral zone (FIG. 13A) and central zone (FIG. 13B) of the tumor. Left bars are before treatment and right bars are after treatment. Treatments, from left to right, are as follows: control; irradiation; irradiation+cisplatin; irradiation+paramagnetic+cisplatin+magnet.

### DETAILED DESCRIPTION OF THE INVENTION

Disclosed herein is the identification of radiation-inducible genes by isolating RNA from irradiated cell cultures and then hybridizing the isolated RNA to nucleic acid sequences from an organism of interest (e.g. mammals such as mice and human beings), such as can optionally be found on microarrays, including but not limited to gene chips. For example, endoglin and carbamyl phosphate synthetase genes have been identified.

Also disclosed herein is a method for x-ray guided drug delivery using a targeting ligand that specifically recognizes a particular radiation-inducible target. For example one embodiment provides a method for x-ray guided delivery to radiation-inducible RNA target molecules using double-stranded RNAs (dsRNAs) as targeting ligands that selectively bind to radiation-induced transcripts.

Also disclosed herein is the magnetic dispersion of an active agent, such as the dispersion of a genetic construct within a tumor. In one embodiment a delivery vehicle comprising a paramagnetic material, such as Fe or Gd, and a genetic construct are administered to a tumor and distributed throughout the tumor by application of external or internal magnetic fields.

I. Definitions

It must be noted that as used herein and in the appended statements of the invention, the singular forms “a”, “and”, and “the” include plural referents unless the context clearly dictates otherwise. Thus, for example, reference to “a construct” includes a plurality of such constructs, and so forth.

The term “about”, as used herein when referring to a measurable value such as an amount of weight, time, dose, etc. is meant to encompass variations of in one embodiment ±20% or ±10%, in another embodiment ±5%, in another embodiment ±1%, and in still another embodiment ±0.1% from the specified amount, as such variations are appropriate to perform the disclosed methods.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All patents and publications mentioned herein are incorporated herein by reference for the purpose of describing and disclosing, for example, the cell lines, constructs, and methodologies that are described in the patents and publications, which might be used in connection with the presently described invention. The patents and publications discussed throughout the text are provided solely for their disclosure prior to the filing date of the present application. Nothing herein is to be construed as an admission that the inventors are not entitled to antedate such disclosure by virtue of prior invention.

While the following terms are believed to be well understood by one of ordinary skill in the art, the following definitions are set forth to facilitate explanation of the invention.

The terms “nucleic acid material” and “nucleic acids” each refer to deoxyribonucleotides, ribonucleotides, or analogues thereof in either single- or double-stranded form. Unless specifically limited, the term encompasses nucleic acids containing known analogues of natural nucleotides that have similar properties as the reference natural or antisense nucleic acid. Thus “nucleic acids” includes but is not limited to DNA, cDNA, RNA, antisense RNA, and double-stranded RNA. A therapeutic nucleic acid can comprise a nucleotide sequence encoding a therapeutic gene product, including a polypeptide or an oligonucleotide.

Nucleic acids can further comprise a gene (e.g., a therapeutic gene), or a genetic construct (e.g., a gene therapy vector). The term “gene” refers broadly to any segment of DNA associated with a biological function. A gene encompasses sequences including but not limited to a coding sequence, a promoter region, a cis-regulatory sequence, a non-expressed DNA segment that is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation of an existing sequence.

The term “expression”, as used herein to describe a genetic construct, generally refers to the cellular processes by which a biologically active polypeptide or biologically active oligonucleotide is produced from a DNA sequence.

The term “construct”, as used herein to describe a genetic construct, refers to a composition comprising a vector used for gene therapy or other application. In one embodiment, the composition also includes nucleic acids comprising a nucleotide sequence encoding a therapeutic gene product, for example a therapeutic polypeptide or a therapeutic oligonucleotide. In one embodiment, the nucleotide sequence is operatively inserted with the vector, such that the nucleotide sequence encoding the therapeutic gene product is expressed. The term “construct” also encompasses a gene therapy vector in the absence of a nucleotide sequence encoding a therapeutic polypeptide or a therapeutic oligonucleotide, referred to herein as an “empty construct.” The term “construct” further encompasses any nucleic acid that is intended for in vivo studies, such as nucleic acids used for triplex and antisense pharmacokinetic studies.

The term “ionizing radiation” is meant to refer to any radiation where a nuclear particle has sufficient energy to remove an electron or other particle from an atom or molecule, thus producing an ion and a free electron or other particle. Examples of such ionizing radiation include, but are not limited to, gamma rays, X-rays, protons, electrons and alpha particles. Ionizing radiation is commonly used in medical radiotherapy and the specific techniques for such treatment will be apparent to a skilled practitioner in the art.

The term “delivery vehicle” as used herein is meant to refer to any cell, molecule, peptide, conjugate, construct, article or other vehicle as would be appreciated by one of ordinary skill in the art after reviewing the present disclosure that can be used to carry an active agent to a target tissue in accordance with the present invention.

The term “active agent” is meant to refer to compounds that are therapeutic agents or imaging agents.

The term “therapeutic agent” is meant to refer to any agent having a therapeutic effect, including but not limited to chemotherapeutics, toxins, radiotherapeutics, or radiosensitizing agents.

The term “chemotherapeutic” is meant to refer to compounds that, when contacted with and/or incorporated into a cell, produce an effect on the cell, including causing the death of the cell, inhibiting cell division or inducing differentiation.

The term “toxin” is meant to refer to compounds that, when contacted with and/or incorporated into a cell, produce the death of the cell.

The term “radiotherapeutic” is meant to refer to radionuclides which when contacted with and/or incorporated into a cell, produce the death of the cell.

The term “radiosensitizing agent” is meant to refer to agents which increase the susceptibility of cells to the damaging effects of ionizing radiation or which become more toxic to a cell after exposure of the cell to ionizing radiation. A radiosensitizing agent permits lower doses of radiation to be administered and still provide a therapeutically effective dose.

The term “imaging agent” is meant to refer to compounds that can be detected.

The term “neoplasm” is meant to refer to an abnormal mass of tissue or cells. The growth of these tissues or cells exceeds and is uncoordinated with that of the normal tissues or cells and persists in the same excessive manner after cessation of the stimuli that evoked the change. These neoplastic tissues or cells show a lack of structural organization and coordination relative to normal tissues or cells that usually result in a mass of tissues or cells that can be either benign or malignant. Representative neoplasms thus include all forms of cancer, benign intracranial neoplasms, and aberrant blood vessels such as arteriovenous malformations (AVM), angiomas, macular degeneration, and other such vascular anomalies. As would be apparent to one of ordinary skill in the art, the term “tumor” typically refers to a larger neoplastic mass.

As used herein, neoplasm includes any neoplasm, including particularly all forms of cancer. This includes, but is not limited to, melanoma, adenocarcinoma, malignant glioma, prostatic carcinoma, kidney carcinoma, bladder carcinoma, pancreatic carcinoma, thyroid carcinoma, lung carcinoma, colon carcinoma, rectal carcinoma, brain carcinoma, liver carcinoma, breast carcinoma, ovary carcinoma, and the like. This also includes, but is not limited to, solid tumors, solid tumor metastases, angiofibromas, retrolental fibroplasia, hemangiomas, Karposi's sarcoma and the like cancers which require neovascularization to support tumor growth.

The phrase “treating a neoplasm” includes, but is not limited to, halting the growth of the neoplasm, killing the neoplasm, reducing the size of the neoplasm, or obliterating a neoplasm comprising a vascular anomaly. Halting the growth of the neoplasm refers to halting any increase in the size of the neoplasm or the neoplastic cells, or halting the division of the neoplasm or the neoplastic cells. Reducing the size of the neoplasm relates to reducing the size of the neoplasm or the neoplastic cells.

The term “subject” as used herein refers to any target of the treatment. Also provided by the present invention is a method of treating neoplastic cells that were grown in tissue culture. Also provided by the present invention is a method of treating neoplastic cells in situ, or in their normal position or location, for example, neoplastic cells of breast or prostate tumors. These in situ neoplasms can be located within or on a wide variety of hosts; for example, human hosts, canine hosts, feline hosts, equine hosts, bovine hosts, porcine hosts, and the like. Any host in which is found a neoplasm or neoplastic cells can be treated and is accordance with the present invention.

The term “subject” as used herein refers to any invertebrate or vertebrate species. The methods of the present invention are particularly useful in the treatment and diagnosis of warm-blooded vertebrates. Thus, the invention concerns mammals and birds. More particularly, provided is the treatment and/or diagnosis of mammals such as humans, as well as those mammals of importance due to being endangered (such as Siberian tigers), of economical importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos, as well as fowl, and more particularly domesticated fowl, e.g., poultry, such as turkeys, chickens, ducks, geese, guinea fowl, and the like, as they are also of economical importance to humans. Thus, provided is the treatment of livestock, including, but not limited to domesticated swine (pigs and hogs), ruminants, horses, poultry, and the like.

The terms “pharmaceutically acceptable”, “physiologically tolerable” and grammatical variations thereof, as they refer to compositions, carriers, diluents and reagents, are used interchangeably and represent that the materials are capable of administration to or upon a vertebrate subject without the production of undesirable physiological effects such as nausea, dizziness, gastric upset and the like.

The term “induce”, as used herein to refer to changes resulting from radiation exposure, encompasses activation of gene transcription or regulated release of proteins from cellular storage reservoirs to vascular endothelium. Alternatively, induction can refer to a process of conformational change, also called activation, such as that displayed by the GPIIb/IIIa integrin receptor upon radiation exposure (Staba et al., 2000; Hallahan et al., 2001). See also U.S. Pat. No. 6,159,443. Irradiated tumors can be targeted using antibodies, peptides, or small molecules that specifically recognize radiation-induced surface proteins as disclosed in Hallahan et al., 2001; Staba et al., 2000; and U.S. Pat. No. 6,159,443.

The term “radiation inducible target” is meant to refer to any target molecule, nucleic acid (including in one embodiment RNA), protein, peptide or other substance whose presence in a target tissue is related to the exposure of the target tissue to ionizing radiation.

The terms “bind”, “binding”, “binding activity” and “binding affinity” are believed to have well-understood meanings in the art. To facilitate explanation of the present invention, the terms “bind” and “binding” are meant to refer to protein-protein interactions that are recognized to play a role in many biological processes, such as the binding between an antibody and an antigen, and between complementary strands of nucleic acids (e.g. DNA-DNA, DNA-RNA, and RNA-RNA). Exemplary protein-protein interactions include, but are not limited to, covalent interactions between side chains, such as disulfide bridges between cysteine residues; hydrophobic interactions between side chains; and hydrogen bonding between side chains.

The terms “binding activity” and “binding affinity” are also meant to refer to the tendency of one protein or polypeptide to bind or not to bind to another protein or polypeptide. The energetics of protein-protein interactions are significant in “binding activity” and “binding affinity” because they define the necessary concentrations of interacting partners, the rates at which these partners are capable of associating, and the relative concentrations of bound and free proteins in a solution. The binding of a ligand to a target molecule can be considered specific if the binding affinity is about 1×10⁴ M⁻¹ to about 1×10⁶ M⁻¹ or greater.

The phrase “specifically (or selectively) binds”, for example when referring to the binding capacity of an antibody, also refers to a binding reaction which is determinative of the presence of the antigen in a heterogeneous population of proteins and other biological materials. The phrase “specifically (or selectively) binds” also refers to selective targeting of a targeting molecule, such as the hybridization of a RNA molecule to a nucleic acid of interest under a set of hybridization conditions as disclosed herein below.

II. Radiation Inducible Targets

In one aspect the identification of radiation-inducible genes by isolating RNA from irradiated cell cultures is disclosed. The isolated RNA is then hybridized to nucleic acid sequences from an organism of interest (e.g. mammals such as mice and human beings) under appropriate conditions. For example, the RNA can be hybridized against a microarray, such as but not limited to a gene chip.

By way of particular example, RNA is isolated from irradiated HUVEV, HMEC, and 3B11 endothelial cells. The RNA is then hybridized to the human and/or mouse gene chips. Fibroblasts have also been utilized to identify radiation-inducible genes. A number of genes are induced in endothelial cells, including endoglin, carbamyl phosphate synthetase, and others.

Endothelial cells and tissues are of particular interest as sources for isolation of RNA. Blood vessels from target tissues, including particularly neoplasm, and more particularly tumors, comprise endothelial tissue. As blood vessels are often targets for delivery of an active agent, endothelial cells and tissues are a source of particular interest. Indeed, optionally, RNA samples are isolated from neoplasm endothelial tissue, and more particularly, tumor endothelial tissue, such as from tumor blood vessels, blood vessels that feed the tumor, and combinations thereof. However, any cell or tissue that is desired to be targeted can be employed as a source of RNA.

II.A. Nucleic Acid Labeling

In one embodiment of the invention, labeling can be carried prior to hybridization. For example, an unlabeled RNA isolated from a biological sample can be detected by hybridization to a labeled nucleic acid from a subject of interest. In another embodiment the RNA is labeled and the nucleic acid from the subject of interest is not labeled. In another embodiment, both the RNA and the nucleic acids include a label, wherein the proximity of the labels following hybridization enables detection. An exemplary procedure using nucleic acids labeled with chromophores and fluorophores to generate detectable photonic structures is described in U.S. Pat. No. 6,162,603 to Heller.

In accordance with the methods of the present invention, any detectable label can be employed. It will be understood to one of skill in the art that any suitable method for labeling can be used, and no particular detectable label or technique for labeling should be construed as a limitation of the disclosed methods.

Direct labeling techniques include incorporation of radioisotopic or fluorescent nucleotide analogues into nucleic acids by enzymatic synthesis in the presence of labeled nucleotides or labeled PCR primers. A radio-isotopic label can be detected using autoradiography or phosphorimaging. A fluorescent label can be detected directly using emission and absorbance spectra that are appropriate for the particular label used. Any detectable fluorescent dye can be used, including but not limited to FITC (fluorescein isothiocyanate), FLUOR X™, ALEXA FLUOR® 488, OREGON GREEN® 488, 6-JOE (6-carboxy-4′,5′-dichloro-2′,7′-dimethoxyfluorescein, succinimidyl ester), ALEXA FLUOR® 532, Cy3, ALEXA FLUOR® 546, TMR (tetramethylrhodamine), ALEXA FLUOR® 568, ROX (X-rhodamine), ALEXA FLUOR® 594, TEXAS RED®, BODIPY® 630/650, and Cy5 (available from Amersham Pharmacia Biotech of Piscataway, N.J., United States of America or from Molecular Probes Inc. of Eugene, Oreg., United States of America). Fluorescent tags also include sulfonated cyanine dyes (available from Li-Cor, Inc. of Lincoln, Nebr., United States of America) that can be detected using infrared imaging. Methods for direct labeling of a heterogeneous nucleic acid sample are known in the art and representative protocols can be found in, for example, DeRisi et al. (1996) Nat Genet 14:457-460; Sapolsky & Lipshutz (1996) Genomics 33:445-456; Schena et al. (1995) Science 270:467-470; Schena et al. (1996) Proc Natl Acad Sci USA 93:10614-10619; Shalon et al. (1996) Genome Res 6:639-645; Shoemaker et al. (1996) Nat Genet 14:450-456; and Wang et al. (1998) Proc Natl Acad Sci USA 86:9717-9721.

Indirect labeling techniques can also be used in accordance with the methods of the present invention, and in some cases, can facilitate detection of rare target sequences by amplifying the label during the detection step. Indirect labeling involves incorporation of epitopes, including recognition sites for restriction endonucleases, into amplified nucleic acids prior to hybridization. Following hybridization, a protein that binds the epitope is used to detect the epitope tag.

In one embodiment, a biotinylated nucleotide can be included in the amplification reactions to produce a biotin-labeled nucleic acid sample. Following hybridization, the label can be detected by binding of an avidin-conjugated fluorophore, for example streptavidin-phycoerythrin, to the biotin label. Alternatively, the label can be detected by binding of an avidin-horseradish peroxidase (HRP) streptavidin conjugate, followed by calorimetric detection of an HRP enzymatic product.

The quality of sample labeling can be approximated by determining the specific activity of label incorporation. For example, in the case of a fluorescent label, the specific activity of incorporation can be determined by the absorbance at 260 nm and 550 nm (for Cy3) or 650 nm (for Cy5) using published extinction coefficients (Randolph & Waggoner (1995) Nuc Acids Res 25:2923-2929). Very high label incorporation (specific activities of >1 fluorescent molecule/20 nucleotides) can result in a decreased hybridization signal compared with probe with lower label incorporation. Very low specific activity (<1 fluorescent molecule/100 nucleotides) can give unacceptably low hybridization signals. See Worley et al. (2000) in Shena, ed., Microarray Biochip Technology, pp. 65-86, Eaton Publishing, Natick, Mass., United States of America. Thus, it will be understood to one of skill in the art that labeling methods can be optimized for performance in microarray hybridization assay, and that optimal labeling can be unique to each label type.

II.B. Microarrays

In one embodiment of the invention, the one or more nucleic acids from the subject of interest are immobilized on a solid support such that a position on the support identifies a particular nucleic acid. In the case of a set, constituent nucleic acids of the set can be combined prior to placement on the solid support or by serial placement of constituent nucleic acid at a same position on the solid support.

A microarray can be assembled using any suitable method known to one of skill in the art, and any one microarray configuration or method of construction is not considered to be a limitation of the present invention. Representative microarray formats that can be used in accordance with the methods of the present invention are described herein below.

II.C. Array Substrate and Configuration

The substrate for printing the array should be substantially rigid and amenable to immobilization and detection methods (e.g., in the case of fluorescent detection, the substrate must have low background fluorescence in the region of the fluorescent dye excitation wavelengths). The substrate can be nonporous or porous as determined most suitable for a particular application. Representative substrates include but are not limited to a glass microscope slide, a glass coverslip, silicon, plastic, a polymer matrix, an agar gel, a polyacrylamide gel, and a membrane, such as a nylon, nitrocellulose or ANAPORE™ (Whatman of Maidstone, United Kingdom) membrane.

Porous substrates (membranes and polymer matrices) permit immobilization of relatively large amount of probe molecules and provide a three-dimensional hydrophilic environment for biomolecular interactions to occur (Dubiley et al. (1997) Nuc Acids Res 25:2259-2265; Yershov et al. (1996) Proc Natl Acad Sci USA 93:4319-4918). A BIOCHIP ARRAYER™ dispenser (Packard Instrument Company of Meriden, Conn., United States of America) can effectively dispense nucleic acids onto membranes such that the spot size is consistent among spots whether one, two, or four droplets were dispensed per spot (Englert (2000) in Schena, ed., Microarray Biochip Technology, pp. 231-246, Eaton Publishing, Natick, Mass., United States of America).

A microarray substrate for use in accordance with the methods of the present invention can have either a two-dimensional (planar) or a three-dimensional (non-planar) configuration. An exemplary three-dimensional microarray is the FLOW-THRU™ chip (Gene Logic, Inc. of Gaithersburg, Md., United States of America), which has implemented a gel pad to create a third dimension. Such a three-dimensional microarray can be constructed of any suitable substrate, including glass capillary, silicon, metal oxide filters, or porous polymers. See Yang et al. (1998) Science 282:2244-2246 and Steel et al. (2000) in Schena, ed., Microarray Biochip Technology, pp. 87-118, Eaton Publishing, Natick, Mass., United States of America.

Briefly, a FLOW-THRU™ chip (Gene Logic, Inc.) comprises a uniformly porous substrate having pores or microchannels connecting upper and lower faces of the chip. Probe nucleic acids are immobilized on the walls of the microchannels and a hybridization solution comprising sample nucleic acids can flow through the microchannels. This configuration increases the capacity for probe and target binding by providing additional surface relative to two-dimensional arrays. See U.S. Pat. No. 5,843,767.

II.D. Surface Chemistry

The particular surface chemistry employed is inherent in the microarray substrate and substrate preparation. Immobilization of nucleic acids probes post-synthesis can be accomplished by various approaches, including adsorption, entrapment, and covalent attachment. Preferably, the binding technique does not disrupt hybridization activity.

For substantially permanent immobilization, covalent attachment is preferred. Since few organic functional groups react with an activated silica surface, an intermediate layer is advisable for substantially permanent probe immobilization. Functionalized organosilanes can be used as such an intermediate layer on glass and silicon substrates (Liu & Hlady (1996) Coll Sur B 8:25-37; Shriver-Lake (1998) in Cass & Ligler, eds., Immobilized Biomolecules in Analysis, pp. 1-14, Oxford Press, Oxford, United Kingdom). A hetero-bifunctional cross-linker requires that the probe have a different chemistry than the surface, and is preferred to avoid linking reactive groups of the same type. A representative hetero-bifunctional cross-linker comprises gamma-maleimidobutyryloxy-succimide (GMBS) that can bind maleimide to a primary amine of a probe. Procedures for using such linkers are known to one of skill in the art and are summarized by Hermanson (1990) Bioconjugate Techniques, Academic Press, San Diego, Calif. A representative protocol for covalent attachment of DNA to silicon wafers is described by O'Donnell et al. (1997) Anal Chem 69:2438-2443.

When using a glass substrate, the glass should be substantially free of debris and other deposits and have a substantially uniform coating. Pretreatment of slides to remove organic compounds that can be deposited during their manufacture can be accomplished, for example, by washing in hot nitric acid. Cleaned slides can then be coated with 3-aminopropyltrimethoxysilane using vapor-phase techniques. After silane deposition, slides are washed with deionized water to remove any silane that is not attached to the glass and to catalyze unreacted methoxy groups to cross-link to neighboring silane moieties on the slide. The uniformity of the coating can be assessed by known methods, for example electron spectroscopy for chemical analysis (ESCA) or ellipsometry (Ratner & Castner (1997) in Vickerman, ed., Surface Analysis: The Principal Techniques, John Wiley & Sons, New York; Schena et al. (1995) Science 270:467-470). See also Worley et al. (2000) in Schena, ed., Microarray Biochip Technology, pp. 65-86, Eaton Publishing, Natick, Mass., United States of America.

For attachment of probe nucleic acids greater than about 300 base pairs, noncovalent binding is suitable. When using this method, amino-silanized slides are preferred in that this coating improves nucleic acid binding when compared to bare glass. This method works well for spotting applications that use about 100 ng/μl (Worley et al. (2000) in Schena, ed., Microarray Biochip Technology, pp. 65-86, Eaton Publishing, Natick, Mass., United States of America).

In the case of nitrocellulose or nylon membranes, the chemistry of nucleic acid binding chemistry to these membranes has been well characterized (Southern (1975) J Mol Biol 98:503-517); Maniatis et al. (1989) Molecular Cloning: A Laboratory Manual, Cold Spring Harbor Press, Cold Spring Harbor, N.Y.).

II.E. Arraying Techniques

A microarray can be constructed using any one of several methods available in the art, including but not limited to photolithographic and microfluidic methods. Of course, ready-made, commercially available microarrays can also be employed.

As is standard in the art, a technique for making a microarray should create consistent and reproducible spots. Each spot is preferably uniform, and appropriately spaced away from other spots within the configuration. A solid support for use in the present invention preferably comprises about 10 or more spots, or more preferably about 100 or more spots, even more preferably about 1,000 or more spots, and still more preferably about 10,000 or more spots. Also preferably, the volume deposited per spot is about 10 picoliters to about 10 nanoliters, and more preferably about 50 picoliters to about 500 picoliters. The diameter of a spot is preferably about 50 μm to about 1000 μm, and more preferably about 100 μm to about 250 μm.

Representative techniques thus include: (1) Light-directed synthesis (Fodor et al. (1991) Science 251:767-773; Fodor et al. (1993) Nature 364:555-556; U.S. Pat. No. 5,445,934; and commercialized by Affymetrix of Santa Clara, Calif., United States of America); (2) Contact Printing (Maier et al. (1994) J Biotechnol 35:191-203; Rose (2000) in Shena, ed., Microarray Biochip Technology, pp. 19-38, Eaton Publishing, Natick, Mass., United States of America; Schena et al. (1995) Science 270:467-470; Mace et al. (2000) in Shena, ed., Microarray Biochip Technology, pp. 39-64, Eaton Publishing, Natick, Mass., United States of America); (3) Noncontact Ink-Jet Printing (U.S. Pat. No. 5,965,352; Theriault et al. (1999) in Schena, ed., DNA Microarrays: A Practical Approach, pp. 101-120, Oxford University Press Inc., New York, N.Y.); (4) Syringe-Solenoid Printing (U.S. Pat. Nos. 5,743,960 and 5,916,524); (5) Electronic Addressing (U.S. Pat. No. 6,225,059 and International Publication No. WO 01/23082); and (6) Nanoelectrode Synthesis (U.S. Pat. No. 6,123,819).

II.E. Hybridization

The terms “specifically hybridizes” and “selectively hybridizes” each refer to binding, duplexing, or hybridizing of a molecule only to a particular nucleotide sequence under stringent conditions when that sequence is present in a complex nucleic acid mixture (e.g., total cellular DNA or RNA).

The phrase “substantially hybridizes” refers to complementary hybridization between a probe nucleic acid molecule and a substantially identical target nucleic acid molecule as defined herein. Substantial hybridization is generally permitted by reducing the stringency of the hybridization conditions using art-recognized techniques.

“Stringent hybridization conditions” and “stringent hybridization wash conditions” in the context of nucleic acid hybridization experiments are both sequence- and environment-dependent. Longer sequences hybridize specifically at higher temperatures. Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. Typically, under “stringent conditions” a probe hybridizes specifically to its target sequence, but to no other sequences.

An extensive guide to the hybridization of nucleic acids is found in Tijssen (1993) Laboratory Techniques in Biochemistry and Molecular Biology-Hybridization with Nucleic Acid Probes, part I chapter 2, Elsevier, New York, N.Y. In general, a signal to noise ratio of 2-fold (or higher) than that observed for a negative control probe in a same hybridization assay indicates detection of specific or substantial hybridization.

II.E.1. Hybridization on a Solid Support

In another embodiment of the invention, a labeled RNA sample is hybridized to one or more nucleic acids that are immobilized on a continuous solid support comprising a plurality of identifying positions. For some high-density glass-based microarray experiments, hybridization at 65° C. is too stringent for typical use, at least in part because the presence of fluorescent labels destabilizes the nucleic acid duplexes (Randolph & Waggoner (1997) Nuc Acids Res 25:2923-2929). Alternatively, hybridization can be performed in a formamide-based hybridization buffer as described in Piétu et al. (1996) Genome Res 6:492-503.

A microarray format can be selected for use based on its suitability for electrochemical-enhanced hybridization. Provision of an electric current to the microarray, or to one or more discrete positions on the microarray facilitates localization of a target nucleic acid sample near probes immobilized on the microarray surface. Concentration of target nucleic acid near arrayed probe accelerates hybridization of a nucleic acid of the sample to a probe. See U.S. Pat. Nos. 6,017,696 and 6,245,508.

II.E.2. Hybridization in Solution

In another embodiment of the invention, a labeled RNA sample is hybridized to one or nucleic acids of interest in solution. Representative stringent hybridization conditions for complementary nucleic acids having more than about 100 complementary residues are overnight hybridization in 50% formamide with 1 mg of heparin at 42° C. An example of highly stringent wash conditions is 15 minutes in 0.1×SSC, 5M NaCl at 65° C. An example of stringent wash conditions is 15 minutes in 0.2×SSC buffer at 65° C. (See Sambrook et al., eds. (1989) Molecular Cloning: A Laboratory Manual, Cold Spring Harbor Laboratory Press, Cold Spring Harbor, N.Y. for a description of SSC buffer). A high stringency wash can be preceded by a low stringency wash to remove background probe signal. An example of medium stringency wash conditions for a duplex of more than about 100 nucleotides, is 15 minutes in 1×SSC at 45° C. An example of low stringency wash for a duplex of more than about 100 nucleotides, is 15 minutes in 4-6×SSC at 40° C. Stringent conditions can also be achieved with the addition of destabilizing agents such as formamide.

For short probes (e.g., about 10 to 50 nucleotides), stringent conditions typically involve salt concentrations of less than about 1M Na⁺ ion, typically about 0.01M to 1M Na⁺ ion concentration (or other salts) at pH 7.0-8.3, and the temperature is typically at least about 30° C.

II.F. Detection

Methods for detecting a hybridization duplex or triplex are selected according to the label employed.

In the case of a radioactive label (e.g., ³²P-dNTP) detection can be accomplished by autoradiography or by using a phosphorimager as is known to one of skill in the art. Preferably, a detection method can be automated and is adapted for simultaneous detection of numerous samples.

Common research equipment has been developed to perform high-throughput fluorescence detecting, including instruments from GSI Lumonics (Watertown, Mass., United States of America), Amersham Pharmacia Biotech/Molecular Dynamics (Sunnyvale, Calif., United States of America), Applied Precision Inc. (Issauah, Wash., United States of America), Genomic Solutions Inc. (Ann Arbor, Mich., United States of America), Genetic MicroSystems Inc. (Woburn, Mass., United States of America), Axon (Foster City, Calif., United States of America), Hewlett Packard (Palo Alto, Calif., United States of America), and Virtek (Woburn, Mass., United States of America). Most of the commercial systems use some form of scanning technology with photomultiplier tube detection. Criteria for consideration when analyzing fluorescent samples are summarized by Alexay et al. (1996) The International Society of Optical Engineering 2705/63.

In another embodiment, labeling with far infrared, near infrared, or infrared fluorescent dyes is employed. Following hybridization, the mixture is scanned photoelectrically with a laser diode and a sensor, wherein the laser scans with scanning light at a wavelength within the absorbance spectrum of the fluorescent label, and light is sensed at the emission wavelength of the label. See U.S. Pat. Nos. 6,086,737; 5,571,388; 5,346,603; 5,534,125; 5,360,523; 5,230,781; 5,207,880; and 4,729,947. An ODYSSEY™ infrared imaging system (Li-Cor, Inc. of Lincoln, Nebr., United States of America) can be used for data collection and analysis.

If an epitope label has been used, a protein or compound that binds the epitope can be used to detect the epitope. For example, an enzyme-linked protein can be subsequently detected by development of a colorimetric or luminescent reaction product that is measurable using a spectrophotometer or luminometer, respectively.

In one embodiment, INVADER® technology (Third Wave Technologies of Madison, Wis., United States of America) is used to detect target nucleic acid/probe complexes. Briefly, a nucleic acid cleavage site (such as that recognized by a variety of enzymes having 5′ nuclease activity) is created on a target sequence, and the target sequence is cleaved in a site-specific manner, thereby indicating the presence of specific nucleic acid sequences or specific variations thereof. See U.S. Pat. Nos. 5,846,717; 5,985,557; 5,994,069; 6,001,567; and 6,090,543.

Surface plasmon resonance spectroscopy can also be used to detect hybridization duplexes formed as disclosed herein. See e.g., Heaton et al. (2001) Proc Natl Acad Sci USA 98(7):3701-3704; Nelson et al. (2001) Anal Chem 73(1):1-7; and Guedon et al. (2000) Anal Chem 72(24):6003-6009.

Numerous software packages have been developed for microarray data analysis, and an appropriate program can be selected according to the array format and detection method. Some products, including ARRAYGAUGE™ software (Fujifilm Medical Systems Inc. of Stamford, Conn., United States of America) and IMAGEMASTER ARRAY 2™0 software (Amersham Pharmacia Biotech of Piscataway, N.J., United States of America), accept images from most microarray scanners and offer substantial flexibility for analyzing data generated by different instruments and array types. Other microarray analysis software products are designed specifically for use with particular array scanners or for particular array formats. A survey of currently available microarray analysis software packages can be found in Brush (2001) The Scientist 15(9):25-28. In addition, the guidance presented herein provides for the development of software and/or databases by one of ordinary skill in the art, to facilitate analysis of data obtained by performing the method of the present invention.

II.G. Delivery of an Active Agent to a Target

The inducible genes also serve as new targets for a delivery vehicle. Antibodies, peptides, and double stranded RNA are provided to bind to the newly expressed RNA. These engineered delivery vehicles can be conjugated to an active agent as defined herein. For example, a radiotherapeutic-immunoconjugate delivery vehicle targeted to radiation-inducible endoglin mRNA can be administered to the subject at an optimal time point following irradiation. The antibody then binds to RNA and carries the radiotherapeutic into the cell, where it binds to RNA.

Double stranded RNA is also referred to as RNA interference (RNAi). See e.g., Zamroe, Nature Structural Biology 8:746, 2001; El Bashir, Nature 411:494, 2001. Methods for using antisense RNA and RNAi, either exogenous addition or transcription in vivo, are known in the art (see Schubiger and Edgar, Methods in Cell Biology (1994) 44:697-713, and PCT application WO 99/32619, respectively. In one embodiment, twenty-one (21)-nucleotide dsRNAs bind to newly transcribed mRNA and can be conjugated to an active agent. The target cells engulf the delivery vehicle comprising the active agent and dsRNAs after administration of the delivery vehicle to a target tissue.

III. Magnetic Dispersion of Active Agents

Also disclosed herein is the magnetic dispersion of an active agent, such as the dispersion of a genetic construct within a target tissue, including but not limited to a neoplasm. In one embodiment a delivery vehicle comprising a paramagnetic material, such as Fe or Gd, and a genetic construct are administered to a tumor and distributed throughout the tumor by application of external or internal magnetic fields. Other representative paramagnetic materials include Co, Ni, Zn, Mn, Mg, Ca, Ba, Sr, Cd, Hg, Al, B, Sc, Ga, V, and In,

Because of the need for improved biodistribution of a genetic construct within a target tissue, disclosed herein are methods and compositions for achieving dispersion. In one embodiment, stable magnetic nanoparticles (also referred to herein as ferrofluids when iron is the paramagnetic material) are used to improve homogeneity of gene therapy within a target tissue. Thus, in one embodiment, ferrofluids are used to deliver gene therapy vectors throughout a tumor microvasculature and to disperse vectors away from needle tracks injected into the tumor.

In one embodiment, magnetic nanoparticles are coated with a targeting agent, such as a targeting agent that binds to irradiated target tissue (e.g. tumor blood vessels), including but not limited to radiation inducible RNA molecules in the irradiated tissue. Other targeting agents are disclosed in U.S. Pat. No. 6,159,443 to Hallahan, and in PCT Publication No. WO 00/66182 (Applicant Vanderbilt University, Inventor Hallahan), herein incorporated by reference.

A targeting molecule can comprise, for example, a ligand that shows specific affinity for a target molecule in the target tissue. See U.S. Pat. Nos. 6,068,829 and 6,232,287. A targeting molecule can also comprise a structural design that mediates tissue-specific localization. For example, extended polymeric molecules can be conjugated to drugs to mediate tumor localization. See U.S. Pat. No. 5,762,909 and the Examples presented below.

Targeting molecules that mediate localization to tumors include in one embodiment ligands that show specific binding to antigens present on tumor vasculature, tumor endothelium (e.g., endothelial cells associated with tumor vasculature), or on tumor cells. For example, a targeting ligand can comprise an antibody or antibody fragment that specifically binds a tumor marker such as Her2/neu (v-erb-b2 avian erythroblastic leukemia viral oncogene homologue 2), CEA (carcinoembryonic antigen), or a ferritin receptor, or that specifically binds to a marker associated with tumor vasculature (integrins, tissue factor, or β-fibronectin isoform). Alternatively, a targeting ligand can comprise a peptide or peptide mimetic that behaves as a tumor homing molecule (Wickham et al., 1995; Staba et al., 2000; International Publication Nos. WO 98/10795 and WO 01/09611; and U.S. Pat. No. 6,180,084).

Radiation-inducible promoters can also be incorporated into the genetic constructs that are dispersed away from the needle tracks in tumors. Using these strategies, biodistribution and bioavailability of therapeutic gene expression in target tissues is markedly improved.

Thus, a method of dispersing a genetic construct in a target tissue is disclosed. In one embodiment, the method comprises: (a) providing a delivery vehicle comprising a genetic construct and a paramagnetic material; (b) administering the delivery vehicle to a target tissue; and (c) applying a magnetic field to the target tissue to thereby disperse the genetic construct.

In another embodiment, a method of enhancing retention of an active agent in a target tissue in a vertebrate subject is disclosed. The method can comprise: (a) providing a delivery vehicle comprising an active agent, a paramagnetic material, and a targeting agent that binds a radiation-induced target molecule; (b) exposing the target tissue to ionizing radiation; (c) exposing the target tissue to a magnetic field; and (d) administering a delivery vehicle to the vertebrate subject, whereby the delivery vehicle localizes to and is retained in the target tissue.

Any suitable paramagnetic material can be employed. Representative embodiments include iron and gadolinium (Fe and Gd respectively). In some cases a further therapeutic effect can be provided through the use of a paramagnetic material that exhibits a photoelectric effect upon interaction with applied ionizing radiation.

The delivery vehicle can further comprise a chemotherapeutic agent, a toxin, a radiotherapeutic agent, a radiosensitizing agent, an imaging agent, and combinations thereof. For example, the biodistribution of particles can be imaged in real time by use of fluoroscopy or MRI. An alternative imaging approach employs radiolabeling vectors, nanoparticles or both vectors and nanoparticles, and imaging by gamma camera during magnetic dispersion.

III.A. Preparation of Magnetic Delivery Vehicles

Ferrofluid particles can be prepared by the methods described by Kuznetsov, A. A., et al., “Ferro-carbon particles: Preparation and chemical applications”, in Hafeli U, Schutt W, Teller J, Zborowski M (Eds), Scientific and Clinical Applications of Magnetic Carriers, Plenum Press, New York (1997). Briefly, iron oxide particles can be formed as follows. Iron oxide precipitates are made by mixing a solution of Fe²⁺ and Fe³⁺ (FeCl₂ and FeCl₃) in NaOH. The precipitate is washed and separated by a magnet until a neutral pH is achieved. Representative ferrofluids include superparamagnetic nanoparticles ranging in size from 5-15 nm of iron oxide (magnetite, Fe₃O₄ or maghemite, Fe₂O₃).

Magnetic particles are encapsulated in various coatings in aqueous media. The resulting ferrofluids are aqueous iron oxide colloids. These magneto-rheological fluids undergo viscosity changes in magnetic field. Stable ferrofluids in physiological media involve the coating of iron oxide particles with dextran (U.S. Pat. No. 4,101,435 to Hasegawa M. S. H.; Dutton A. H., et al., Proc Natl Acad Sci 76:3392-96, 1979; Molday R S, Mackenzie D., J. Immunol. Meth. 52:353-67, 1982; PCT Int. Appl. WO 8303426 to Schroder, 1983; European Patent 0016552 A1 to Widder, K. J., 1980) albumin (European Patent 0420186 A2 to Masahisa O., 1990; U.S. Pat. No. 4,695,392 to Whitehead R. A. 1987; Renshaw P. F., Magn. Reson. Med. 3:217-25, 1986) and other polymers (Ugelstad J. et al., Advances in biomagnetic separation, Eaton Publishing, Natick, Mass., 1994; Int'l Patent WO 91/09141 to Wang, et al. 1991, Arshady R., Biomaterials 14:5-15, 1993). A coating process is also disclosed in U.S. Pat. No. 5,248,492 to Groman. Iron oxide can also be conjugated to DMSA and/or SPDP to stabilize the ferrofluid (French Patent 9006-484 to Bee et al., 1990). See also (Menager C., et al., J. Colloid. Interface Sci. 169:251, 1995; Massart R, et al., Brazilian J. Phys. 25:135-141, 1995; Neveu-Prin S, et al., J Magn Magn. Mat. 122:42-45, 1993; European Patent 9003120 to Neveu-Prin S. et al., 1990; Bacri, J. C., et al., Mat. Sci. Eng. C2:197-203, 1995; Fabre P, et al., Phys. Rev. Lett. 64:530-33, 1990).

Ligands such as proteins can conjugated onto maghemite particles by the heterobifunctional agent SPDP (Carlsson J, et al., Biochem J. 173:723, 1978). Briefly, SPDP is first coupled to the ligand to form an amide bond and the resulting conjugate is linked to the particle by the SH group forming a disulfide bridge (Massart R, et al., Brazilian J. Phys. 25:135-141, 1995). A molar ratio of one ligand grafted to one meghemite particle is typically used.

Magnetite-dextran nanocapsules can be coated with a number of polymers, including but not limited to albumin, polysiloxane, starch, monoclonal antibodies, IgG, PEKY, lipids, carboxy-dextran, and combinations thereof. The biodistribution of these polymer-coated ferrofluids include tumors and lymph nodes. Lipid-coated ferrofluids can be achieved through a variety of techniques (Chan T W, et al., Invest. Radiol. 27:443-49, 1992; Patrizio, G., et al: Cancer targeted liposomes containing superparamagnetic iron oxide: ferrosomes. Proc 8th Annual Meeting of the Society of Magnetic Resonance in Medicine (SMRM), Amsterdam, Berkeley:327,1989).

Another representative polymer coat is, which has a terminal carboxyl group performing covalent bonds with ligands. Siloxane ferrofluids have been designed for radioimmunoassay. (Turner R. D., et al, J Urol. 113:455-59, 1975).

Polystyrene coated maghemite particles are made by chelating polystyrene nanoparticles to a solution of iron salts, followed by precipitation of iron oxide on the particles (Saini S, et al., Radiology 162:211-16, 1987). Magnetite-starch microcapsules can achieve a small particle size of 200 nm (Fahlvik A. K., et al., Invest Radiol 25:113-20, 1990).

Magnetite-dextran nanocapsules are prepared by a method introduced by Whitehead (U.S. Pat. No. 4,554,088; CA 102, P58899r, 1985). Aqueous solution of FeCl₃ and FeCl₂ is added to 16% NH₄OH containing dextran. Alternatively, a procedure by Molday can be used to produce ferromagnetic microcapsules (Molday R. S., et al., J. Immunol. Meth. 52:353-67, 1982). This method produces magnetite-dextran nanocapsules in the 100 nm range.

The delivery vehicle can comprises a linker that links the paramagnetic material and the genetic construct. A variety of linkers can be conjugated to a magnetic nanoparticle. These include chelators or haptens, such as NTA, EDTA, DTPA, and HEDTA. These chelators bind metals, such as but not limited nickel or zinc. These metals form a weak interaction with modified genetic constructs that include inserted peptides, such as polyhystidine and zinc fingers.

Polyethylene glycol can be conjugated to the ferrofluids contained within liposomes. In this embodiment magnetic nanoparticles attached to liposomes can contain genetic constructs. Other therapeutic agents including viral vectors and oncolytic viruses can then be added to liposomes.

Avidin or streptavidin can be conjugated to magnetic nanoparticles to act as linkers. Optionally, the genetic constructs are then biotinylated by a 1:1 molar ratio so that 1 biotin is present on each construct. Biotinylated vectors are then added to the avidin-conjugated magnetic nanoparticles.

Protein A can be conjugated to magnetic nanoparticles. The second step is use of IgG that binds to a vector protein coat, such as the fiber on adenovirus. A 1:1 molar ratio of antibody to vector is typically used. The antibodies bound to vector can then be added to the protein A-conjugated magnetic nanoparticles. Vector can be bound directly to magnetic nanoparticles. One example is the adenovirus vector modified with polylysine (available under the trademark Pk7 from GenVec, Inc. of Gaithersburg, Md., United States of America). This vector adheres to proteins including albumin, protein A, avidin or any other ligand proteins. Alternatively, a polyarginine peptide can be linked to the magnetic nanoparticles so that polylysine will adhere to this peptide. Alternatively, polyhistidine is added to the gene product of the genetic construct, which can then bind Ni-coated ferrofluids.

Bispecific antibodies can also be used as linkers. In this approach, bispecific antibody to coated nanoparticles such as albumin or other ligands bind at one end, and the other end of the antibody binds the genetic construct.

A combined approach of biotinylated components can be used as a linker. In one embodiment, an antibody to vector, such as an antibody to an adenoviral fiber, is biotinylated. The magnetic nanoparticles are conjugated to avidin. The genetic construct is then linked to avidin by use of the biotinylated anti-vector antibody.

In another embodiment, annexin V is conjugated to a magnetic nanoparticle. Annexin V binds to cardiolipin. Cardiolipin can be conjugated to a genetic construct, and the linker is provided by the interaction between annexin and cardiolipin.

In one embodiment, a linker allows the genetic construct to be shed from the ligand as the nanoparticle is pulled through the target tissue. That is, the linker is a cleavable linker, as can be provided by through a particular peptide sequence, among other options. This also allows for the vector to transduce the target cells. When employed to deliver a genetic construct to a target tissue, a magnetic particle can be employed to aid in either directing the therapeutic agent to a target tissue or, as disclosed in other embodiments of the present invention, to disperse the particles away from an administration site (e.g. a needle track). When the structure reaches a desired location, the linker can then be cleaved, thereby releasing the genetic construct, which can then transduce the target cells.

In another embodiment, ferrofluids or other magnetic nanoparticles are added to cells, which act as carriers and/or targeting agents in the delivery vehicles. These cells can include cells that bind within irradiated tumors such as endothelial progenitor cells. These cells bind and extravasate within irradiated tumors. Ferrofluids or other magnetic nanoparticles can also be added to producer cells, such as 293 cells or any cell transduced with a genetic construct (optionally comprising a sequence encoding a therapeutic polypeptide) ex vivo. Another embodiment employs magnetic bacteria comprising a genetic construct, with optional additional therapeutic agent or agents.

III.B. Application of Magnetic Fields

External, internal, and both external and internal magnetic field can be employed dispersion of drug delivery systems, including particularly genetic constructs. External and internal magnets with a large gradient (Tesla/Meter) can be applied to the target tissue. For example, magnets can be placed within afterloading catheters used during brachytherapy.

Thus, genetic constructs and other active agents linked to magnetic nanoparticles are then administered by any suitable route, including but not limited to intravascular, intraarterial, and intravesicular (peritoneum, bladder, gastrointestinal tract, or intratumoral) routes, and combinations thereof. Intratumoral administration can include any approach, such as but not limited to endoscopy, bronchoscopy, proctoscopy, or any fiber optic tool for administration. The magnetic field is then applied for a brief period of time, such as 7 minutes, to disperse the genetic construct linked to the magnetic nanoparticle.

A typical example is the use of transrectal ultrasound to identify rectal tumors or prostate tumors. Gene therapy is then administered by use of needles placed into the tumor. The gene therapy is then pulled away from the needle track by either an internal or external magnetic field, or combination thereof. For example, needles placed in parallel alignment in a tumor can include drug administration and an internal magnet.

An alternative approach can be employed in the treatment of cervical carcinoma. This approach utilizes afterloading devices, such as tandem and ovoids. The vector-linked nanoparticles can be administered into the tandem and magnets can be placed into the ovoids to pull the gene therapy throughout the tumor.

Another approach is the use of bronchoscopy to administrator vectors linked in nanoparticles. An external magnet can be used to pull the vector into the lung tumor.

III.C. Representative Embodiments of Magnetic Delivery Vehicles

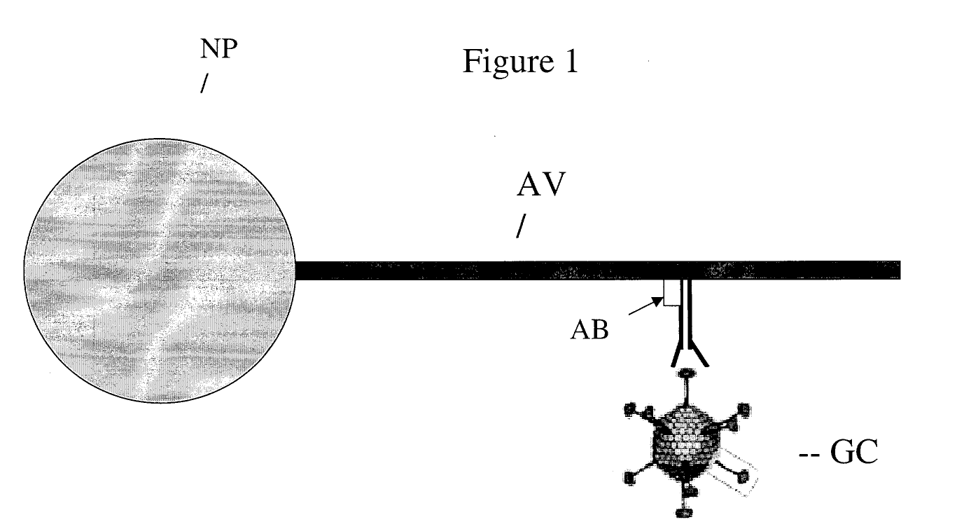

Referring to FIG. 1, a dextran-coated magnetic nanoparticle NP conjugated to avidin AV is linked to genetic construct GC by use of biotinylated construct GC and biotinylated anti-adenovirus antibody AB. Thus, the linker is provided by the interaction between construct GC, antibody AB, and avidin AV. Nanoparticles NP are coated with lectin, which adheres to irradiated tumor blood vessels. Construct GC is pulled into a tumor following intravenous administration. Construct GC then adheres to tumor blood vessels. Expression genes such as beta-galactosidase and green fluorescence protein are then detected.

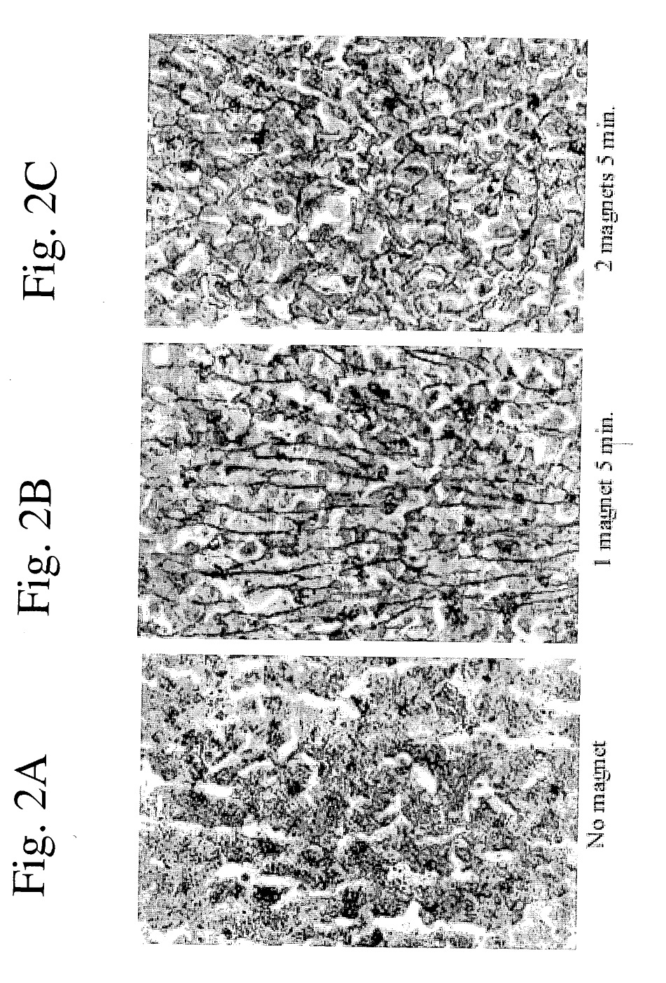

Referring to FIGS. 2A-2C, an adenoviral-beta-galactosidase expression vector (Ad.LacZ) was linked to magnetic nanoparticles as described in FIG. 1. Nanoparticles were dispersed in tumor tissue by use of an external magnet. The vector transduced tumor endothelium. LacZ expression is shown in tumor endothelium. FIG. 2A shows the tissue prior to application of the magnetic field; FIG. 2B shows the tissue after a five minute application of a magnetic field from one magnet; and FIG. 2C shows the tissue after a five minute application of a magnetic field from two magnets.

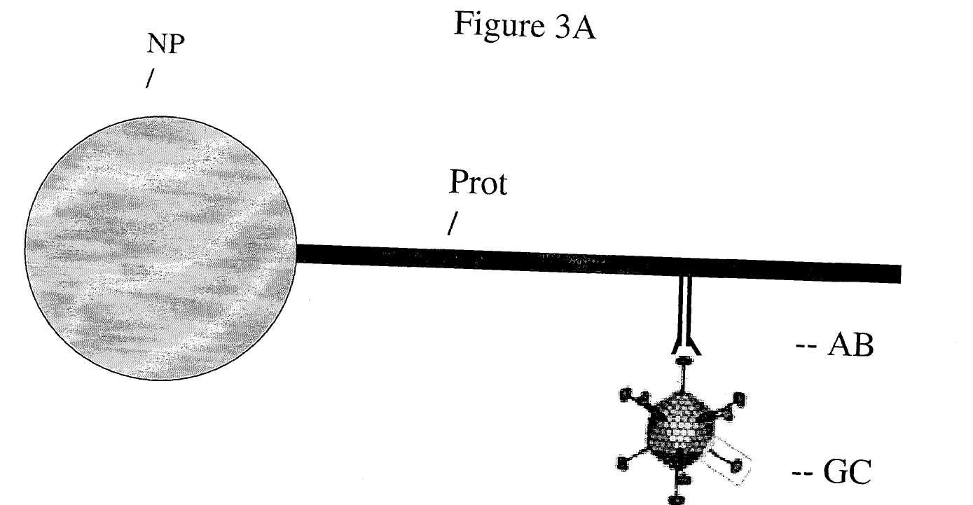

Referring to FIGS. 3A and 3B, an antibody AB to a fiber protein in an adenoviral construct GC is linked to protein coating Prot coated on a magnetic nanoparticle NP. Representative protein coatings include Protein A and albumin. Optionally, antibody AB is a bispecific antibody. Thus, the linker is provided by the interaction between construct GC, antibody AB, and protein coating Prot.

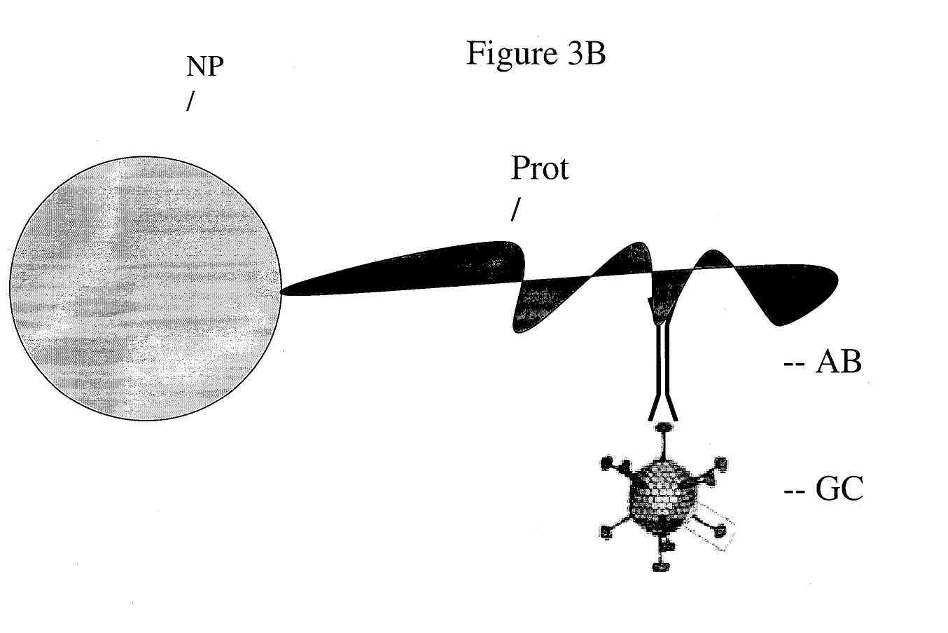

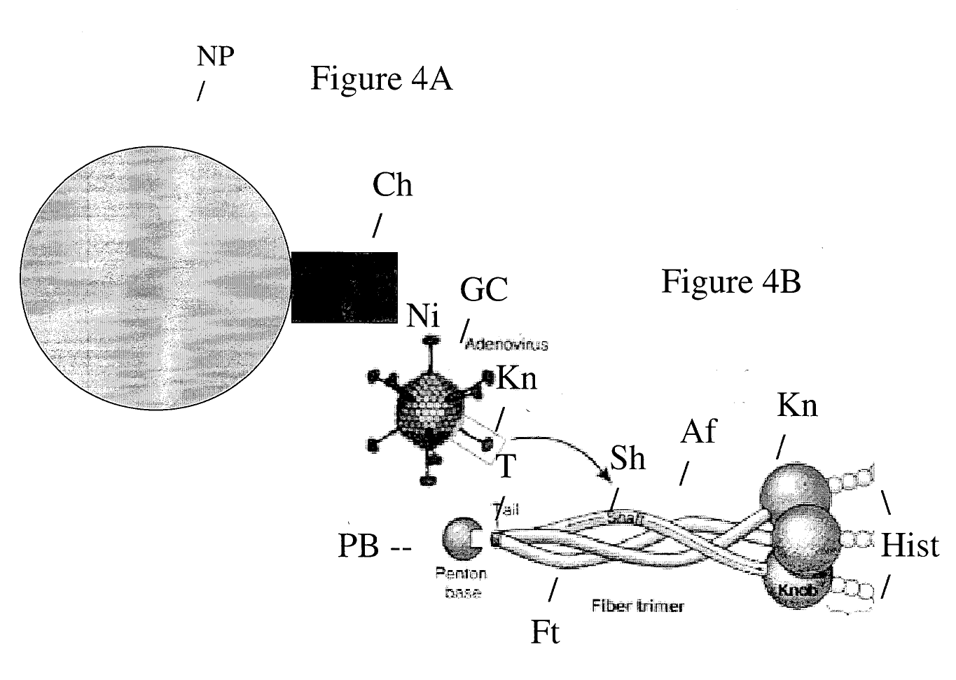

Referring to FIGS. 4A and 4B, an adenoviral fiber Af typically comprises a penton base PB, and a trimer Ft with a tail T and shaft Sh that links base PB to three knobs Kn (also seen in FIGS. 5A and 5B). Polyhistidine Hist is incorporated into knob Kn on a fiber Af of an adenoviral construct GC. Polyhistidine Hist binds to nickel Ni, which is conjugated to a magnetic nanoparticle NP by a chelator Ch, such as DTPA or NTA. Thus, the linker is provided by the interaction between construct GC, polyhistidine Hist, and chelator Ch.

Referring to FIGS. 5A and 5B, polylysine Lys is incorporated into knob Kn on a fiber Af of an adenoviral construct GC. Polylysine Lys binds to a protein Prot that is coated onto magnetic nanoparticle NP. Representative proteins include albumin or protein A. Alternatively, the coated protein Prot can comprise a peptide linker, such as polyarginine. Optionally, the linker is a cleavable linker, such as a peptide sequence having a known cleavage site. Thus, the linker is provided by the interaction between construct GC, polylysine Lys, and protein coating Prot.

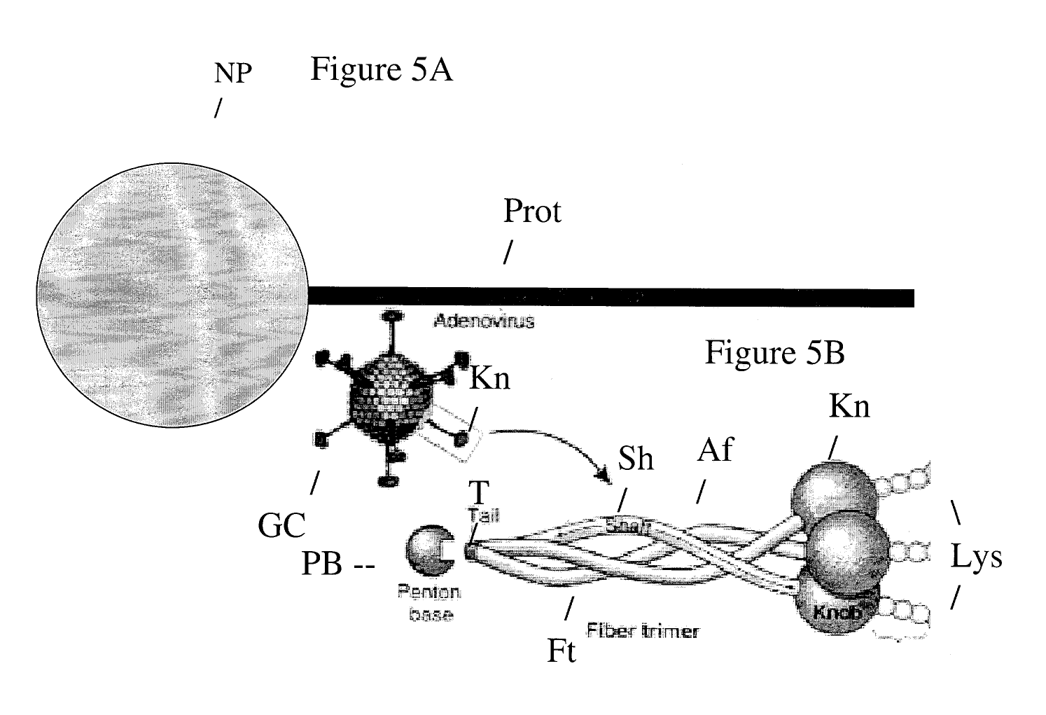

Referring to FIG. 6, a magnetic nanoparticle NP is conjugated to polyethylene glycol PEG, and then incorporated into a liposome LS. A genetic construct GC is then added to liposome LS. Thus, the linker is provided by polyethylene glycol PEG and liposome LS.

IV. Active Agents

A delivery vehicle as disclosed herein can comprise an active agent, such as a therapeutic or an imaging agent. The therapeutic agent can comprise a genetic construct, a chemotherapeutic agent, a toxin, a radiotherapeutic, or a radiosensitizing agent. Each agent is loaded in a total amount effective to accomplish the desired result in the target tissue, whether the desired result be imaging the target tissue or treating the target tissue.

IV.A. Genetic Constructs

A genetic construct optionally comprises a nucleic acid sequence encoding a polypeptide. The genetic construct can comprises an enhancer-promoter region that is responsive to radiation, and expression of the polypeptide is controlled by the enhancer-promoter. The genetic construct further comprises a viral vector.

Genetic constructs can be used for the treatment of any condition wherein expression of a gene product having therapeutic or prophylactic activity is sought. Such constructs are particularly suited for treatment of tumors or other neoplasms.

Representative therapeutic oligonucleotides include, but are not limited to antisense RNA (Ehsan & Mann, 2000; Phillips et al., 2000), double-stranded oligodeoxynucleotides (Morishita et al., 2000), ribozymes (Shippy et al., 1999; de Feyter & Li, 2000; Norris et al., 2000; Rigden et al., 2000; Rossi, 2000; Smith & Walsh, 2000; Lewin & Hauswirth, 2001), and peptide nucleic acids (Ehsan & Mann, 2000; Phillips et al., 2000). Methods for the design, preparation, and testing of therapeutic oligonucleotides can be found in the sources listed herein above, and references cited therein, among other places.

Representative therapeutic polypeptides include those polypeptides that are abnormally absent or expressed at insufficient levels in a subject. A therapeutic polypeptide can also comprise a polypeptide that is antagonistic to an abnormal activity in a subject, for example unregulated cell division. For example, compositions useful for cancer therapy include, but are not limited to genes encoding tumor suppressor gene products/antigens, antimetabolites, suicide gene products, anti-angiogenesis agents, immunostimulatory agents, and combinations thereof, as described further herein below. See generally Kirk & Mule, 2000; Mackensen et al., 1997; Walther & Stein, 1999; and references cited therein.

In one embodiment of the invention, genetic constructs are used for cancer therapy. Angiogenesis and a suppressed immune response play central roles in the pathogenesis of malignant disease and tumor growth, invasion, and metastasis. Thus, therapeutic nucleic acids encode in one embodiment polypeptides, in another embodiment oligonucleotides, and in another embodiment peptide-nucleic acids having an ability to induce an immune response and/or an anti-angiogenic response in vivo.

The term “immune response” is meant to refer to any response to an antigen or antigenic determinant by the immune system of a vertebrate subject. Exemplary immune responses include humoral immune responses (e.g. production of antigen-specific antibodies) and cell-mediated immune responses (e.g. lymphocyte proliferation).

Representative therapeutic proteins with immunostimulatory effects include but are not limited to cytokines (e.g., IL-2, IL-4, IL-7, IL-12, interferons, granulocyte-macrophage colony-stimulating factor (GM-CSF), tumor necrosis factor alpha (TNF-α)), immunomodulatory cell surface proteins (e.g., human leukocyte antigen (HLA proteins), co-stimulatory molecules, and tumor-associated antigens. See Kirk & Mule, 2000; Mackensen et al., 1997; Walther & Stein, 1999; and references cited therein.

The term “angiogenesis” refers to the process by which new blood vessels are formed. The term “anti-angiogenic response” and “anti-angiogenic activity” as used herein, each refer to a biological process wherein the formation of new blood vessels is inhibited.

Representative proteins with anti-angiogenic activities that can be used in accordance with the present invention include: thrombospondin I (Kosfeld & Frazier, 1993; Tolsma et al., 1993; Dameron et al., 1994), metallospondin proteins (Carpizo & Iruela-Arispe, 2000), class I interferons (Albini et al., 2000), IL-12 (Voest et al., 1995), protamine (Ingber et al., 1990), angiostatin (O'Reilly et al., 1994), laminin (Sakamoto et al., 1991), endostatin (O'Reilly et al., 1997), and a prolactin fragment (Clapp et al., 1993). In addition, several anti-angiogenic peptides have been isolated from these proteins (Maione et al., 1990; Eijan et al., 1991; Woltering et al., 1991).

In one embodiment of the invention, an anti-angiogenic polypeptide comprises Tie-2, an endothelium-specific receptor tyrosine kinase (Lin et al., 1998b). Endogenous ligands are bound by ectopically expressed Tie-2, and signaling via the endogenous Tie-2 receptor to promote tumor growth is thereby blocked.

In another embodiment of the invention, an anti-angiogenic polypeptide comprises a soluble form of vascular endothelial growth factor (VEGF) receptor, more preferably the Flk-1 receptor. The soluble VEGF receptors can function as dominant negative inhibitors of VEGF signaling and have been used to promote tumor regression. See Goldman et al., 1998; Takayama et al., 2000; Lin et al., 1998a; and PCT International Publication No. WO 00/37502.

A gene therapy construct used in accordance with the methods of the present invention can also encode a therapeutic gene that displays both immunostimulatory and anti-angiogenic activities, for example, IL-12 (Dias et al., 1998; and references cited herein below), interferon-α (O'Byrne et al., 2000, and references cited therein), or a chemokine (Nomura & Hasegawa, 2000, and references cited therein). In addition, a gene therapy construct can encode a gene product with immunostimulatory activity and a gene product having anti-angiogenic activity. See e.g., Narvaiza et al., 2000.

IV.A.2. Promoters

A gene therapy construct of the invention can employ any suitable promoter, including both constitutive promoters, inducible promoters, and tissue-specific promoters. Representative inducible promoters include chemically regulated promoters (e.g., the tetracycline-inducible expression system, Gossen & Bujard, 1992; Gossen & Bujard, 1993; Gossen et al., 1995), a radiosensitive promoter (e.g., the egr-1 promoter, Weichselbaum et al, 1994; Joki et al., 1995; the E-selectin promoter, Hallahan et al, 1995a), and heat-responsive promoters (Csermely et al., 1998; Easton et al., 2000; Ohtsuka & Hata, 2000). Representative tissue-specific promoters include the CEA promoter, which is selectively expressed in cancer cells (Hauck & Stanners, 1995; Richards et al., 1995).

IV.A.3. Vectors

The genetic constructs of the present invention comprise vectors that facilitate transduction and expression of the gene therapy construct in a host cell. The particular vector employed in accordance with the disclosed methods is not necessarily intended to be a limitation of the methods disclosed herein.

The term “vector” as used herein refers to a nucleic acid molecule having nucleotide sequences that enable its replication in a host cell. A vector can also include nucleotide sequences to permit ligation of nucleotide sequences within the vector, wherein such nucleotide sequences are also replicated in a host cell. Representative vectors comprising nucleic acids include plasmids, cosmids, and viral vectors.

The term “vector” also includes non-nucleic acid compositions that can facilitate introduction of nucleic acids into a host cell, for example a liposome. As described further herein below, constructs comprising non-nucleic acid vectors are prepared by encapsulating or otherwise associating nucleic acids having nucleotide sequences that enable its replication in a host cell.

Any suitable vector for delivery of the genetic construct can be used including, but not limited to viruses, plasmids, water-oil emulsions, polyethylene imines, dendrimers, micelles, microcapsules, liposomes, and cationic lipids, or other appropriately lipid, micelle or liposome having an appropriate charge or polarity. Representative vectors that are amenable to the targeting and dispersion methods disclosed herein include viral vectors, plasmids, and liposomes, each described further herein below. Where appropriate, two or more types of vectors can be used together. For example, a plasmid vector can be used in conjunction with liposomes. See e.g., U.S. Pat. No. 5,928,944.

Suitable methods for introduction of the vector into cells include direct injection into a cell or cell mass, particle-mediated gene transfer, hyper-velocity gene transfer, electroporation, DEAE-Dextran transfection, liposome-mediated transfection, viral infection, and combinations thereof. A delivery method is selected based considerations such as the vector type, the toxicity of the encoded gene, and the condition to be treated.

Viral Gene Therapy Vectors. Representative viruses for gene transfer include, but are not limited to adenoviruses (Zwiebel et al., 1998; Hitt & Graham, 2000; Silman & Fooks, 2000), adeno-associated virus (Halbert et al., 1995; Guha et al., 2000; Tal, 2000; Smith-Arica & Bartlett, 2001), herpes simplex virus (e.g. herpes simplex virus type 1) (Cunningham & Davison, 1993; Yeung & Tufaro, 2000; Latchman, 2001), RNA negative strand viruses (e.g., mumps virus) (Palese et al., 1996), parvovirus (Srivastava, 1994; Shaughnessy et al., 1996), Epstein-Barr virus (Delecluse & Hammerschmidt, 2000; Komaki & Vos, 2000), alphaviruses (e.g., Sindbis virus and Semliki virus) (Lundstrom, 1999; Wahlfors et al., 2000), baculovirus (Sandig et al., 1996; Sarkis et al., 2000), retroviruses (Cruz et al., 2000b; Cruz et al., 2000a), polyoma and papilloma viruses (Krauzewicz & Griffin, 2000), and varicella-zoster virus (Cohen & Seidel, 1993). Methods for preparation of viral vectors for gene therapy can be found in the above-cited sources, and references cited therein, among other places.

Viral vectors are in one embodiment replication-deficient. That is, they lack one or more functional genes required for their replication, which prevents their uncontrolled replication in vivo and avoids undesirable side effects of viral infection. In one embodiment, all of the viral genome is removed except for the minimum genomic elements required to package the viral genome incorporating the therapeutic gene into the viral coat or capsid. For example, it is desirable to delete all the viral genome except the Long Terminal Repeats (LTRs) or Invented Terminal Repeats (ITRs) and a packaging signal. In the case of adenoviruses, deletions are typically made in the E1 region and optionally in one or more of the E2, E3 and/or E4 regions. In the case of retroviruses, genes required for replication, such as env and/or gag/pol can be deleted. Deletion of sequences can be achieved using recombinant techniques, for example, involving digestion with appropriate restriction enzymes, followed by religation. Replication-competent self-limiting or self-destructing viral vectors can also be used.

Nucleic acid constructs of the invention can be incorporated into viral genomes by any suitable technique known in the art. Typically, such incorporation will be performed by ligating the construct into an appropriate restriction site in the genome of the virus.

Viral genomes can then be packaged into viral coats or capsids by any suitable procedure. In particular, any suitable packaging cell line can be used to generate viral vectors of the invention. These packaging lines complement the replication-deficient viral genomes of the invention, as they include, typically incorporated into their genomes, the genes which have been deleted from the replication-deficient genome. Thus, the use of packaging lines allows viral vectors of the invention to be generated in culture. For example, suitable packaging lines for retroviruses include derivatives of PA317 cells, ψ-2 cells, CRE cells, CRIP cells, E-86-GP cells, and 293GP cells. Line 293 cells can be used for adenoviruses and adeno-associated viruses. Neuroblastoma cells can be used for herpes simplex virus, e.g. herpes simplex virus type 1.

The term “helper cell” as used herein refers to a cell that is transduced with a genetic construct or a vector, wherein the helper cell can amplify the genetic construct or vector. Thus, the term “helper cell” includes prokaryotic, eukaryotic, and plant heterologous expression systems. The term “helper cell” also encompasses packaging cells used to prepare viral vectors, as described further herein below.

In one embodiment of the invention, a genetic construct comprises a viral vector. In one embodiment, a viral vector of the invention is disabled, e.g. helper-dependent. The term “helper-dependent” refers to a recombinant viral vector that is incapable of propagation in the absence of a helper functions. Thus, a helper-dependent viral vector typically comprises a deleted and/or altered genome, wherein one or more gene functions required for viral propagation are disrupted. For example, a representative helper-dependent adenoviral vector can comprise functional deletions in one or more of the adenovirus genes E2a, E4, the late genes L1 through L5, and/or the intermediate genes IX and IVa.

The terms “packaging cell” or “packaging cell line” refer to a cell line that permits or facilitates virus replication and packaging. A packaging cell line typically comprises trans-complementing functions that have been deleted from a helper-dependent virus. Suitable packaging lines for retroviruses include derivatives of PA317 cells, ψ-2 cells, CRE cells, CRIP cells, E-86-GP cells, and 293GP cells. Line 293 cells can be used for adenoviruses and adeno-associated viruses.

Plasmid Gene Therapy Vectors. A gene therapy construct of the present invention can also include a plasmid. Advantages of using plasmid vectors include low toxicity and relatively simple large-scale production. A major obstacle that has prevented the widespread application of plasmid DNA is its relative inefficiency in gene transduction. Electroporation has been used to effectively transport molecules including DNA into living cells in vitro (Neumann et al., 1982). Recent reports have demonstrated the use of electroporation in vivo, for example to enhance local efficiency of chemotherapeutic agents (Hofmann et al., 1999; Sersa et al., 2000).

Plasmid transfection efficiency in vivo encompasses a multitude of parameters, such as the amount of plasmid, time between plasmid injection and electroporation, temperature during electroporation, and electrode geometry and pulse parameters (field strength, pulse length, pulse sequence, etc.). The methods disclosed herein can be optimized for a particular application by methods known to one of skill in the art, and the present invention encompasses such variations. See e.g., Heller et al., 1996; Vicat et al., 2000; and Miklavcic et al., 1998.

Liposomes. The present invention also envisions the use of gene therapy constructs comprising liposomes. Representative liposomes include, but are not limited to cationic liposomes, optionally coated with polyethylene glycol (PEG) to reduce non-specific binding of serum proteins and to prolong circulation time. See Koning et al., 1999; Nam et al., 1999; and Kirpotin et al., 1997. Temperature-sensitive liposomes can also be used, for example THERMOSOMES™ as disclosed in U.S. Pat. No. 6,200,598. A gene therapy construct can further comprise plasmid—liposome complexes as described in U.S. Pat. No. 5,851,818.

Liposomes can also be prepared by any of a variety of techniques that are known in the art. See e.g., Betageri et al., 1993; Gregoriadis, 1993; Janoff, 1999; Lasic & Martin, 1995; Nabel, 1997; and U.S. Pat. Nos. 4,235,871; 4,551,482; 6,197,333; and 6,132,766. As one example, PEG 2000-PE, cholesterol, Dipalmitoyl phosphocholine (Avanti® Polar Lipids, Inc., Alabaster, Ala., United States of America), Dil (lipid fluorescent marker available from Molecular Probes, Inc., Eugene, Oreg., United States of America), and maleimide-PEG-2000-DOPE are dissolved in chloroform and mixed at a ratio of 10:43:43:2:2 in a round bottom flask as described in Leserman et al., 1980. The organic solvent is removed by evaporation followed by desiccation under vacuum for 2 hours. Liposomes are prepared by hydrating the dried lipid film in phosphate-buffered saline at a lipid concentration of 10 mM. The suspension is then sonicated 3×5 minutes until clear, forming unilamellar liposomes of 100 nm in diameter.

Entrapment of an active agent within liposomes can be carried out using any conventional method in the art. In preparing liposome compositions, stabilizers such as antioxidants and other additives can be used (Leserman, 1980; Betageri et al., 1993; Gregoriadis, 1993; Lasic & Martin, 1995; Nabel, 1997; Janoff, 1999).

Other lipid carriers can also be used in accordance with the claimed invention, such as lipid microparticles, micelles, sphingosomes, lipid suspensions, and lipid emulsions. See e.g., Labat-Moleur et al., 1996 and U.S. Pat. Nos. 5,011,634; 5,814,335; 6,056,938; 6,217886; 5,948,767; and 6,210,707.

IV.B. Other Active Agents

Chemotherapeutics useful as active agents are typically small chemical entities produced by chemical synthesis. Chemotherapeutics include cytotoxic and cytostatic drugs. Chemotherapeutics can include those which have other effects on cells such as reversal of the transformed state to a differentiated state or those which inhibit cell replication. Exemplary chemotherapeutic agents include, but are not limited to, anti-tumor drugs, cytokines, anti-metabolites, alkylating agents, hormones, and the like.

Additional examples of chemotherapeutics include common cytotoxic or cytostatic drugs such as for example: methotrexate (amethopterin), doxorubicin (adrimycin), daunorubicin, cytosine arabinoside, etoposide, 5-4 fluorouracil, melphalan, chlorambucil, and other nitrogen mustards (e.g. cyclophosphamide), cis-platinum, vindesine (and other vinca alkaloids), mitomycin and bleomycin. Other chemotherapeutics include: purothionin (barley flour oligopeptide), macromomycin, 1,4-benzoquinone derivatives, trenimon, steroids, aminopterin, anthracyclines, demecolcine, etoposide, mithramycin, doxorubicin, daunomycin, vinblastine, neocarzinostatin, macromycin, α-amanitin and the like. Certainly, the use of combinations of chemotherapeutic agents is also provided.

Toxins are useful as active agents. Toxins are generally complex toxic products of various organisms including bacteria, plants, etc.

Exemplary toxins include, but are not limited to, coagulants such as Russell's Viper Venom, activated Factor IX, activated Factor X or thrombin; and cell surface lytic agents such as phospholipase C, (Flickinger & Trost, Eu. J. Cancer 12(2):159-60 (1976)) or cobra venom factor (CVF) (Vogel & Muller-Eberhard, Anal. Biochem 118(2):262-268 (1981)) which should lyse neoplastic cells directly. Additional examples of toxins include but are not limited to: ricin, ricin A chain (ricin toxin), Pseudomonas exotoxin (PE), diphtheria toxin (DT), bovine pancreatic ribonuclease (BPR), pokeweed antiviral protein (PAP), abrin, abrin A chain (abrin toxin), gelonin (GEL), saporin (SAP), modeccin, viscumin and volkensin.

Exemplary radiotherapeutic agents include, but are not limited to, ⁴⁷Sc, ⁶⁷Cu, ⁹⁰Y, ¹⁰⁹Pd, ¹²³I, ¹²⁵I, ¹³¹I, ¹¹¹In, ¹⁸⁶Re, ¹⁸⁸Re, ¹⁹⁹Au, ²¹¹At, ²¹²Pb and ²¹²Bi. Other radionuclides which have been used by those having ordinary skill in the art include: ³²P and ³³P, ⁷¹Ge, ⁷⁷As, ¹⁰³Pb, ¹⁰⁵Rh, ¹¹¹Ag, ¹¹⁹Sb, ¹²¹Sn, ¹³¹Cs, ¹⁴³Pr, ¹⁶¹Tb, ¹⁷⁷Lu, ¹⁹¹Os, ^{193M}Pt, ¹⁹⁷Hg, all beta negative and/or auger emitters. Some preferred radionuclides include: ⁹⁰Y, ¹³¹I, ²¹¹At and ²¹²Pb/²¹²Bi.

Radiosensitizing agents are substances that increase the sensitivity of cells to radiation. Exemplary radiosensitizing agents include, but are not limited to, nitroimidazoles, metronidazole and misonidazole (see DeVita, V. T. Jr. in Harrison's Principles of Internal Medicine, p. 68, McGraw-Hill Book Co., N.Y. 1983, which is incorporated herein by reference), as well as art-recognized boron-neutron capture and uranium capture systems. See, e.g., Gabe, D. Radiotherapy & Oncology 30:199-205 (1994); Hainfeld, J. Proc. Natl. Acad. Sci. USA 89:11064-11068 (1992). A delivery vehicle comprising a radiosensitizing agent as the active moiety is administered and localizes at the target tissue. Upon exposure of the tissue to radiation, the radiosensitizing agent is “excited” and causes the death of the cell.

Radiosensitizing agents are also substances which become more toxic to a cell after exposure of the cell to ionizing radiation. In this case, DNA protein kinase (PK) inhibitors, such as R106 and R116 (ICOS, Inc.); tyrosine kinase inhibitors, such as SU5416 and SU6668 (Sugen Inc.); and inhibitors of DNA repair enzymes comprise examples.

Another provided radiosensitizing agent comprises a genetic construct that comprises an enhancer-promoter region that is responsive to radiation, and at least one nucleic acid encoding a polypeptide whose expression is controlled by the enhancer-promoter. In accordance with the present invention, methods of destroying, altering, or inactivating cells in target tissue by delivering the genetic constructs to the cells of the tissues via delivery vehicles and inducing expression of the structural gene or genes in the construct by exposing the tissues to ionizing radiation are also provided. Such genetic constructs are loaded, conjugated or otherwise linked with a delivery vehicle as described herein above. Exemplary genetic constructs and related techniques are described in U.S. Pat. Nos. 5,817,636; 5,770,581; 5,641,755; and 5,612,318, the entire contents of each of which herein incorporated by reference.

Exemplary imaging agents include, but are not limited to, paramagnetic, radioactive and fluorogenic ions. Preferably, the imaging agent comprises a radioactive imaging agent. Exemplary radioactive imaging agents include, but are not limited to, gamma-emitters, positron-emitters and x-ray-emitters. Particular radioactive imaging agents include, but are not limited to, ⁴³K, ⁵²Fe, ⁵⁷Co, ⁶⁷Cu, ⁶⁷Ga, ⁶⁸Ga, ⁷⁷Br, ⁸¹Rb/^{81M}Kr, ^{87m}Sr, ^{99m}Tc, ¹¹¹In, ¹¹³In, ¹²³I, ¹²⁵I, ¹²⁷Cs, ¹²⁹Cs, ¹³¹I, ¹³²I, ¹⁹⁷Hg, ²⁰³Pb and ²⁰⁶Bi. Other radioactive imaging agents known by one skilled in the art can be used as well.

IV.C. Dosages for Active Agents

For therapeutic applications, a therapeutically effective amount of a composition of the invention is administered to a subject. A “therapeutically effective amount” is an amount of the therapeutic composition sufficient to produce a measurable biological response (including, but not limited to an immunostimulatory response, an anti-angiogenic response, a cytotoxic response, or tumor regression). Actual dosage levels of active ingredients in a therapeutic composition of the invention can be varied so as to administer an amount of the active compound(s) that is effective to achieve the desired therapeutic response for a particular subject and/or application. The selected dosage level will depend upon a variety of factors including, but not limited to the activity of the therapeutic composition, formulation, the route of administration, combination with other drugs or treatments, severity of the condition being treated (e.g., in the case of a tumor, tumor size and longevity), and the physical condition and prior medical history of the subject being treated. In one embodiment, a minimal dose is administered, and dose is escalated in the absence of dose-limiting toxicity. Determination and adjustment of a therapeutically effective dose, as well as evaluation of when and how to make such adjustments, are known to those of ordinary skill in the art of medicine.

For diagnostic applications, a detectable amount of a composition of the invention is administered to a subject. A “detectable amount”, as used herein to refer to a diagnostic composition, refers to a dose of such a composition that the presence of the composition can be determined in vivo or in vitro. A detectable amount will vary according to a variety of factors, including, but not limited to chemical features of the drug being labeled, the detectable label, labeling methods, the method of imaging and parameters related thereto, metabolism of the labeled drug in the subject, the stability of the label (e.g. the half-life of a radionuclide label), the time elapsed following administration of the drug and/or labeled antibody prior to imaging, the route of drug administration, and the physical condition and prior medical history of the subject. Thus, a detectable amount can vary and can be tailored to a particular application. After study of the present disclosure, it is within the skill of one in the art to determine such a detectable amount.

For local administration of viral vectors, previous clinical studies have demonstrated that up to 10¹³ pfu of virus can be injected with minimal toxicity. In human patients, 1×10⁹-1×10¹³ pfu are routinely used. See Habib et al., 1999. To determine an appropriate dose within this range, preliminary treatments can begin with 1×10⁹ pfu, and the dose level can be escalated in the absence of dose-limiting toxicity. Toxicity can be assessed using criteria set forth by the National Cancer Institute and is reasonably defined as any grade 4 toxicity or any grade 3 toxicity persisting more than 1 week. Dose can also be modified to maximize anti-tumor and/or anti-angiogenic activity. Representative criteria and methods for assessing anti-tumor and/or anti-angiogenic activity are described herein below.

For the purposes of cell therapy, cells (e.g. cells for ex vivo therapy) can be delivered by injection in one embodiment and by subcutaneous administration in another embodiment. A person of skill in the art will be able to choose an appropriate dosage, e.g. the number and concentration of cells, to take into account the fact that only a limited volume of fluid can be administered in this manner.

Because delivery vehicles are specifically targeted to target tissues, a delivery vehicle that comprises an active agent is typically administered in a dose less than that which is used when the active agent is administered directly to a subject, preferably in doses that contain up to about 100 times less active agent. In some embodiments, delivery vehicles that comprise an active agent are administered in doses that contain about 10 to about 100 times less active agent as an active moiety than the dosage of active agent administered directly. To determine the appropriate dose, the amount of compound is preferably measured in moles instead of by weight. In that way, the variable weight of delivery vehicles does not affect the calculation. A one to one ratio of delivery vehicle to active agent in the delivery vehicles of the present invention is presumed.

Typically, chemotherapeutic conjugates are administered intravenously in multiple divided doses. Up to 20 gm IV/dose of methotrexate is typically administered. When methotrexate is administered as the active moiety in a delivery vehicle of the invention, there is about a 10- to 100-fold dose reduction. Thus, presuming each delivery vehicle includes one molecule of methotrexate to one mole of delivery vehicle, of the total amount of delivery vehicle active agent administered, up to about 0.2 to about 2.0 g of methotrexate is present and therefore administered. In some embodiments, of the total amount of delivery vehicle/active agent administered, up to about 200 mg to about 2 g of methotrexate is present and therefore administered.

By way of further example, doxorubicin and daunorubicin each weigh about 535. Presuming each delivery vehicle includes one molecule of doxorubicin or daunorubicin to one delivery vehicle, a provided dose range for delivery vehicle-doxorubicin vehicle or delivery vehicle-daunorubicin is between about 40 to about 4000 mg. In some embodiments, dosages of about 100 to about 1000 mg of delivery vehicle-doxorubicin or delivery vehicle-daunorubicin are administered. In some embodiments, dosages of about 200 to about 600 mg of delivery vehicle-doxorubicin or delivery vehicle-daunorubicin are administered.

Toxin-containing loaded delivery vehicles are formulated for intravenous administration. Using an intravenous approach, up to 6 nanomoles/kg of body weight of toxin alone have been administered as a single dose with marked therapeutic effects in patients with melanoma (Spitler L. E., et al. (1987) Cancer Res. 47:1717). In some embodiments of the present invention, then, up to about 11 micrograms of delivery vehicle-toxin/kg of body weight may be administered for therapy.

The molecular weight of ricin toxin A chain is 32,000. Thus, for example, presuming each delivery vehicle includes one molecule of ricin toxin A chain to one delivery vehicle, delivery vehicles comprising ricin toxin A chain are administered in doses in which the proportion by weight of ricin toxin A chain is about 1 to about 500 μg of the total weight of the administered dose. In some preferred embodiments, delivery vehicles comprising ricin toxin A chain are administered in doses in which the proportion by weight of ricin toxin A chain is about 10 to about 100 μg of the total weight of the administered dose. In some preferred embodiments, delivery vehicles comprising ricin toxin A chain are administered in doses in which the proportion by weight of ricin toxin A chain is about 2 to about 50 μg of the total weight of the administered dose.

The molecular weight of diphtheria toxin A chain is 66,600. Thus, presuming each delivery vehicle includes one molecule of diphtheria toxin A chain to one delivery vehicle, delivery vehicles comprising diphtheria toxin A chain are administered in doses in which the proportion by weight of diphtheria toxin A chain is about 1 to about 500 μg of the total weight of the administered dose. In some preferred embodiments, delivery vehicles comprising diphtheria toxin A chain are administered in doses in which the proportion by weight of diphtheria toxin A chain is about 10 to about 100 μg of the total weight of the administered dose. In some preferred embodiments, delivery vehicles comprising diphtheria toxin A chain are administered in doses in which the proportion by weight of diphtheria toxin A chain is about 40 to about 80 μg of the total weight of the administered dose.

The molecular weight of Pseudomonas exotoxin is 22,000. Thus, presuming each delivery vehicle includes one molecule of Pseudomonas exotoxin to one delivery vehicle, delivery vehicles comprising Pseudomonas exotoxin are administered in doses in which the proportion by weight of Pseudomonas exotoxin is about 0.01 to about 100 μg of the total weight of the loaded delivery vehicle-exotoxin administered. In some preferred embodiments, delivery vehicles comprising Pseudomonas exotoxin are administered in doses in which the proportion by weight of Pseudomonas exotoxin is about 0.1 to about 10 μg of the total weight of the administered dose. In some embodiments, delivery vehicles comprising Pseudomonas exotoxin are administered in doses in which the proportion by weight of Pseudomonas exotoxin is about 0.3 to about 2.2 μg of the total weight of the administered dose.

To dose delivery vehicles comprising radioisotopes in pharmaceutical compositions useful as imaging agents, it is presumed that each delivery vehicle is loaded with one radioactive active moiety. The amount of radioisotope to be administered is dependent upon the radioisotope. Those having ordinary skill in the art can readily formulate the amount of delivery vehicle-imaging agent to be administered based upon the specific activity and energy of a given radionuclide used as an active moiety. Typically, about 0.1 to about 100 millicuries per dose of imaging agent, about 1 to about 10 millicuries, or about 2 to about 5 millicuries are administered.

Thus, compositions that are useful imaging agents comprise delivery vehicles comprising a radioactive moiety in an amount ranging from about 0.1 to about 100 millicuries, in some embodiments about 1 to about 10 millicuries, in some embodiments about 2 to about 5 millicuries, in some embodiments about 1 to about 5 millicuries.

Examples of dosages include: ¹³¹I=between about 0.1 to about 100 millicuries per dose, in some embodiments about 1 to about 10 millicuries, in some embodiments about 2 to about 5 millicuries, and in some embodiments about 4 millicuries; ¹¹¹In=between about 0.1 to about 100 millicuries per dose, in some embodiments about 1 to about 10 millicuries, in some embodiments about 1 to about 5 millicuries, and in some embodiments about 2 millicuries, ^{99m}Tc=between about 0.1 to about 100 millicuries per dose, in some embodiments about 5 to about 75 millicuries, in some embodiments about 10 to about 50 millicuries, and in some embodiments about 27 millicuries.

To load delivery vehicles with radioisotopes in compositions useful as therapeutic agents, it is presumed that each delivery vehicle is loaded with one radioactive active moiety. The amount of radioisotope to be administered is dependent upon the radioisotope. Those having ordinary skill in the art can readily formulate the amount of delivery vehicle-radio-therapeutic agent to be administered based upon the specific activity and energy of a given radionuclide used as an active moiety. For therapeutics that comprise ¹³¹I, between about 10 to about 1000 nanomoles (nM), preferably about 50 to about 500 nM, more preferably about 300 nM of ¹³¹I at the tumor, per gram of tumor, is desirable. Thus, if there is about 1 gram of tumor, and about 0.1% of the administered dose is delivered to the tumor, about 0.5 to about 100 mg of ¹³¹I-delivery vehicle is administered. In some embodiments, about 1 to about 50 mg of ¹³¹I-delivery vehicle is administered. In some embodiments, about 5 to about 10 mg of ¹³¹I-delivery vehicle is administered. Wessels B. W. and R. D. Rogus (1984) Med. Phys. 11:638 and Kwok, C. S. et al. (1985) Med. Phys. 12:405, both of which are incorporated herein by reference, disclose detailed dose calculations for diagnostic and therapeutic vehicles which can be used in the preparation of compositions that include radioactive delivery vehicles.

IV.D. Pharmaceutically Acceptable Formulations

After a sufficiently purified delivery vehicle comprising active agent has been prepared, one will desire to prepare it into a pharmaceutically acceptable formulation that can be administered in any suitable manner. Preferred administration techniques include parenteral administration, intravenous administration and injection and/or infusion directly into a target tissue, such as a solid tumor or other neoplastic tissue. This is done by using for the last purification step a pharmaceutically acceptable medium.

Representative compositions generally comprise an amount of the desired delivery vehicle-active agent in accordance with the dosage information set forth above admixed with an acceptable pharmaceutical diluent or excipient, such as a sterile aqueous solution, to give an appropriate final concentration in accordance with the dosage information set forth above with respect to the active agent. Such formulations will typically include buffers such as phosphate buffered saline (PBS), or additional additives such as pharmaceutical excipients, stabilizing agents such as BSA or HSA, or salts such as sodium chloride.

For parenteral administration it is generally desirable to further render such compositions pharmaceutically acceptable by insuring their sterility, non-immunogenicity and non-pyrogenicity. Such techniques are generally well known in the art as exemplified by Remington's Pharmaceutical Sciences, 16th Ed. Mack Publishing Company (1980), incorporated herein by reference. It should be appreciated that endotoxin contamination should be kept minimally at a safe level, for example, less that 0.5 ng/mg protein. Moreover, for human administration, preparations should meet sterility, pyrogenicity, general safety and purity standards as required by FDA Office of Biological Standards.

### EXAMPLES

The following Examples have been included to illustrate modes of the invention. Certain aspects of the following Examples are described in terms of techniques and procedures found or contemplated by the present co-inventors to work well in the practice of the invention. These Examples illustrate standard laboratory practices of the co-inventors. In light of the present disclosure and the general level of skill in the art, those of skill will appreciate that the following Examples are intended to be exemplary only and that numerous changes, modifications, and alterations can be employed without departing from the scope of the invention.

### Overview of Examples

In the following examples, wheat germ agglutinin (WGA, a lectin) was conjugated to nanoparticle magnetic beads and served as an anchor for particle adhesion while flowing through irradiated tumor blood vessels. Acute inflammation within tumor vasculature was induced by external irradiation, and WGA binds to inflamed vascular endothelium. This delivery vehicle was used to carry cisplatin to the vasculature of three mouse tumor models, LLC, GL-261 and H460.

The magnetic nanoparticles were used as a vehicle to carry compounds or proteins and be guided by a magnetic force field. Once the external magnetic field is removed, the particles re-enter the circulation. An aspect of this embodiment is that the use of a radiation inducible target improved the duration of binding of this delivery vehicle in the target tissue.

### Materials and Methods for Examples

Tumor Model: The LLC and H460 cell lines were obtained from American Type Culture Collection (Manassas, Va., United States of America). The GL-261 cell line was obtained from Dr. Yancie Gillespie (University of Alabama-Birmingham, Birmingham, Alabama, United States of America) (Staba, M. J., Hallahan, D. E. and Weichselbaum, R., Gene Ther. 5: 293-300 (1998); Hallahan, D. E., et al., Cancer Res. 58:5216-5220 (1998)).

LLC and GL-261 cell lines form tumor in C57BL6J mice following subcutaneous injection into either the hind limb or the dorsal skin fold window chamber. H460 cell line forms tumor in nude mice. Cells were trypsinized from cell culture and counted by hemocytometer. 10⁶ cells suspension in complete medium were injected subcutaneously into the hind limb, or 10⁵ cells into dorsal skin fold window.

Tumor Vascular Window Model: 10⁵ cells were injected into the dorsal ventral window. The techniques and procedures were the same as described by Geng, L., et al., Cancer Res. 61:2413-2419 (2001). Tumor blood vessels were developed in the window within 1 week and ready to be used at 7-10 days.

### Example 1

### WGA Binding To Inflamed Endothelial Cells

LLC tumor models were used in this Example. When the tumor size reached to 12-15 mm of diameter the tumors were irradiated with 0 Gy and 3 Gy with the rest of the body shielded by a piece of lead. 100 μg/100 μl of WGA-Biotin (Vector, Burlingame, Calif., United States of America) in PBS was injected into the blood flow through tail vein for each mouse at thirty minutes after irradiation. Thirty minutes later the experimental animals were sacrificed and the 10 μm of tumor frozen sections were cut for biotin staining. 10 μg/ml of Avidin-FITC (Vector, Burlingame, Calif., United States of America) in PBS was used to stain WGA-Biotin on tumor sections in dark at room temperature (RT) for 45 minutes. Slides were washed with PBS for 3 times and 5 μg/ml of DAPI (Sigma Chemical Company, St. Louis, Mo., United States of America) in PBS was used for a counter staining for 5 minutes. PBS:glycerol (1:3) was used as mounting medium and coverslides were mounted. The slides were checked with a fluorescent microscopy and the periphery areas (with more vasculature) of tumor were photographed.

When the LLC window tumor models were ready the windows were irradiated with 0 Gy and 2.5 Gy with the rest of the body shielded by a piece of lead. Thirty minutes later, 100 μl of WGA labeled with FITC were injected by tail vein. The WGA-FITC was made from 60 μl of 1 mg/ml WGA-Biotin (Vector), 40 ul of 5 mg/ml Avidin-FITC (Vector), and 300 μl of PBS mixed well at RT for 30 minutes before using. Fluorescent microscopy pictures of tumor windows were taken 60 minutes after the tail vein injection with the WGA-FITC cocktail.

### Example 2

### Assessement Of Nanomag Particles Binding In The Vasculature Of Tumor

LLC mouse tumor models were used in this Example. When the tumor reached about 15 mm of diameter the experimental animal was taken x-rayed with a X-ray machine (GE SENOGRAPHE™ 600T, SENIX™ HF, at 26 kv and 5 mAs) under anesthesia. Then the same animal was injected with 300 μl of nanomag beads (DTPA surface, 130 nm, 10 mg/ml, Micromod, Germany) into the blood flow by tail vein. The leg with tumor was immediately put in a magnetic force field after the injection and kept there for fifteen minutes, and the second x-ray image film was taken. The magnetic force field was formed with two pieces of 1.26″×0.66″×0.39″ neodymium high power magnets (Edmund Scientific; Tonawanda, N.Y., United States of America) that were fixed on a wooden board with a 20 mm space between of them.

### Example 3

### IHC Stain for WGA

LLC and GL-261 mouse tumor models were used in this Example. When the tumor reached about 15 mm of diameter, 100 μl of 0.5 mg of nanomag-10 μg of WGA (nanomag-DTPA, 130 nm, 10 mg/ml, Micromod, Germany; WGA, Vector, Burlingame, Calif., United States of America) in PBS was injected into the blood flow of tumor-bearing mouse by tail vein. The leg with tumor was immediately put in the magnetic force field after the injection and stayed there for 15 minutes. Then, the experimental animals were sacrificed and the tumors were fixed for a paraffin sections.

Five (5) μm of sections were cut and stained for WGA with immunohistochemistry technique. Briefly, slides were incubated with biotinylated goat anti-WGA antibody (Vector) at 37° C. for 30 minutes, following with vector alkaline phosphatase standard kit and substrate kit to turn the antigen (WGA) blue. 1% of Eosin in 95% of alcohol was used as a counter stain. The WGA (a protein) conjugation to DTPA (nanomag functionalized surface was achieved by activation of the carboxyl group of DTPA with active ester, which was formed by reaction of 1-ethyl-3 [3-(dimethylamino) propyl] carbodiimide (EDC) (Sigma) with N-hydroxysuccinimide (Sigma) (Lewis, M. R., et al., Bioconjug Chem 1994 November-December; 5(6):565-76; Drabick, J. J., et al., Antimicrobial Agents and Chemotherapy, March 1998, 583-88). The unbinding chemical reagents were removed by a simple procedure: pulling the nanomag beads to the bottom of the tube by a magnet and aspirating the supernatant, adding fresh PBS, and washing an additional three times.

### Example 4

### Tumor Volume Assessment

Forty mice bearing GI-261 tumors on their right hind limbs were divided eight groups (five mice per group). An equal number of large and intermediate size (11-15 mm) tumors were present in each group. The first group received no treatment as the control group. The second group received radiation therapy (3 Gy×2 fractions) on days 1 and 3. Irradiated mice were immobilized in LUCITE™ chambers and the entire body was shielded with lead, except for tumor bearing hind limb. The third group received 0.08 mg/100 μl of cisplatin (Sigma) in PBS by tail vein injection on days 1, 2, 3 and 4. The forth group received 0.5 mg of nanomag beads-10 μg of WGA-0.08 mg cisplatin cocktail in 100 μl PBS by tail vein injection on days 1, 2, 3 and 4. The fifth group received irradiation of 3 Gy on days 1 and 3 and the same treatments as group 3 after irradiation on days 1, 2, 3 and 4. The sixth group received irradiation of 3 Gy on days 1 and 3 and the same treatments as group 4 after irradiation on days 1, 2, 3 and 4. The seventh group received the same treatments as group 6 and added a 15 minute treatment of magnetic force field for the leg with tumor each time after injection on days 1, 2, 3 and 4. The eighth group received the same treatments as group 7, except injection cocktail did not contain 10 μg of WGA.

Twenty mice bearing H460 tumors with a range of 10-13 mm of diameter on their right hind limbs were divided into four groups (five mice per group). The first group received no treatment as the control group. The second group received radiation therapy (3 Gy×2 fraction) on days 1 and 3. The third group received radiation as group 1 and 0.08 mg/100 μl of cisplatin in PBS by tail vein injection after irradiation on days 1, 2, 3 and 4. The forth group received radiation as group 1 and 0.5 mg of nanomag beads-10 μg of WGA-0.08 mg of cisplatin cocktail in 100 μl PBS by tail vein injection after irradiation on days 1, 2, 3 and 4 with 15 minutes of magnetic force field after each time injection.

Tumors volumes were measured 3 times weekly using skim calipers as described previously (see e.g., Hallahan, D. E., et al., Nat. Med. 1:786-791 (1995); Hanson, et al., Radiation Research 142:281-287 (1995)) starting on day 1 and ending on the eighth measurement, or when the tumor volume reached 5 times that of the beginning volumes. Data was calculated as the percentage of original (day 1) tumor volume and graphed as fractional tumor volume+/−standard deviation (SD) for each treatment group.

The nanomag beads-WGA-cisplatin cocktail were made from nanomag-DTPA (130 nm, 10 mg/ml, Micromod, Germany), WGA (Vector), 1 μg/ul stock solution in PBS and cisplatin (Sigma), 16 mg/ml stock solution in dimethylformamide (DMF; Sigma). The WGA conjugation to nanomag was carried out as described above and cisplatin binding to the nanomag beads was carried by DTPA chelating to Pt (platinum) contained in cisplatin.

### Example 5

### Power Doppler Sonograph

Power Doppler was used to quantify blood flow of the experimental tumors as described previously by Geng, L., et al., Cancer Res. 61:2413-2419 (2001). The measurements were performed two times on day 1 before treatments started and day 4 after treatments. The blood flow change in peripheral and central zone of the tumors were most interested and collected for a further assay.

### Results of Examples

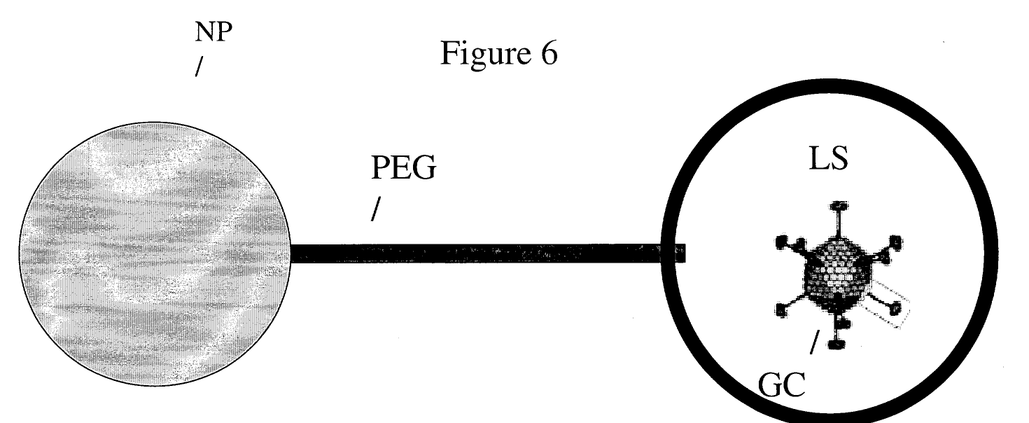

The frozen sections of LLC tumors stained with DAPI and Avidin-FITC show that irradiated tumors have a greater binding of biotin-WGA injected via tail vein (FIGS. 7A and 7B). When the endothelial cells were triggered into an inflammatory reaction with 3 Gy, the lectin binding spots on endothelial cells were exposed to WGA-Biotin that stained by Avidin-FITC.

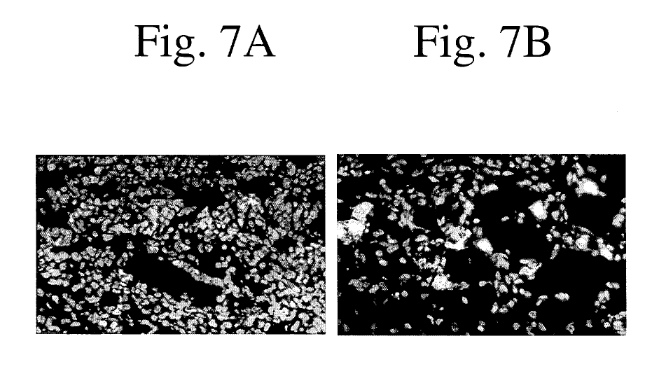

LLC tumor window models were used to test WGA binding to inflamed endothelial cells, which show directly WGA-FITC binding on blood vessels (FIG. 8A, brighter spots). When the vasculature of tumor was irradiated with 2.5 Gy and resulted an acute inflammatory reaction of blood vessels, there was a much better WGA-FITC binding to vasculature (FIG. 8A) as compared to tumors without irradiation (FIG. 8B).

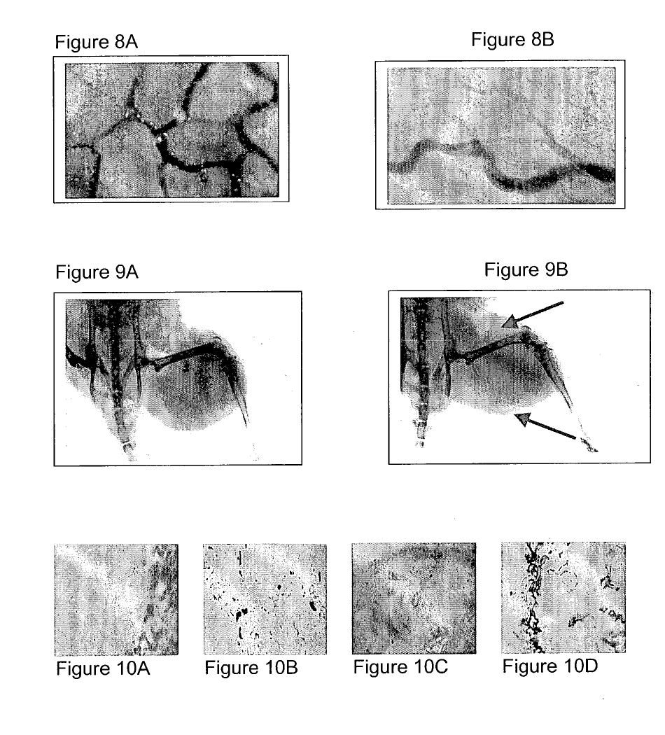

FIGS. 9A and 9B show the x-ray image of blood vessels of LLC tumor before (FIG. 9A) and after (FIG. 9B) injection with nanomag beads and exposed to magnetic force field. The magnetic force drew the beads with iron particles to blood vessels after injected into the blood flow. The arrows indicate the x-ray high-density images on FIG. 9B are two blood vessels that do not show on FIG. 9A, which is the image before injection with beads. After the X-rays were taken the mouse was sacrificed. A homogenizer was used to break down 0.5 g of fresh tumor tissue in PBS.

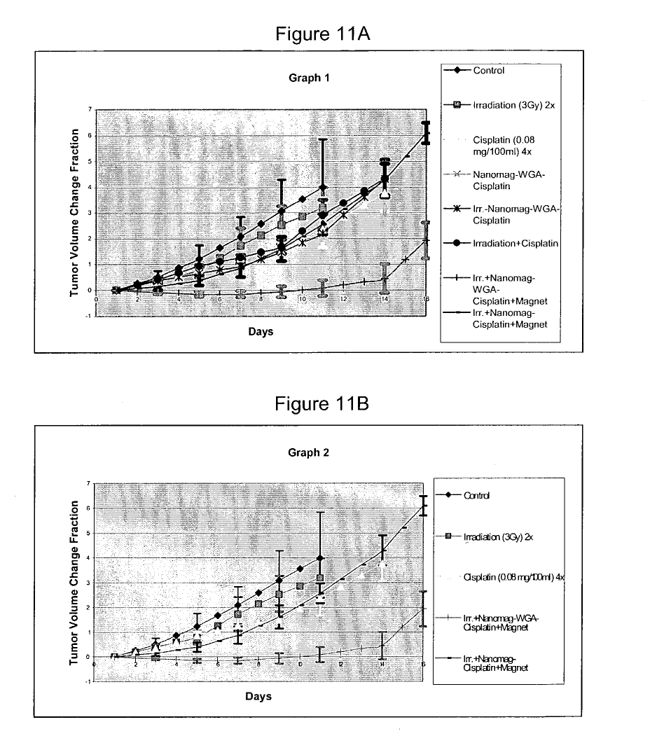

FIGS. 10A-10D show immunohistochemistry staining of GL-261 and LLC tumor sections with magnet and without magnet after injection of nanomag-DTPA-WGA. WGA was stained by goat anti-WGA antibody with alkaline phosphatase image system (darker areas). The magnetic force induced the accumulation of particles within the tumor tissue.

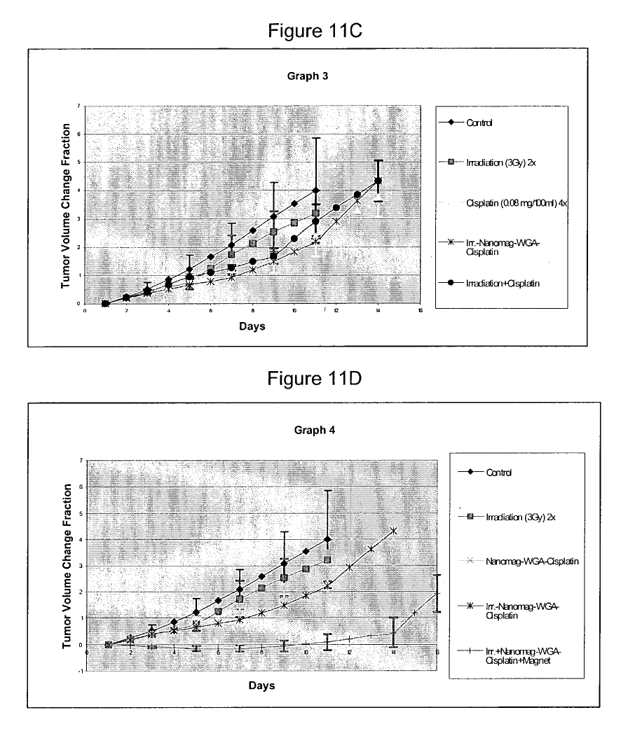

GL-261 tumor volume curves (FIGS. 11A-11D) show a significant difference from the different treatments groups. Two fractions of 3 Gy had no effect on the tumor growth control. Four dosages of cisplatin delayed LLC tumor growth about 3-4 days. The group 4 (nanomag-WGA-cisplatin) had a similar effect to cisplatin alone (group 3).

Similar results were obtained from group 5 and group 6, in which 2 fractions of 3 Gy were added over that administered to group 3 and group 4, respectively. Two irradiation doses did not increase apparent effects on both of the treatment groups. The irradiation+nanomag-WGA-cisplatin+magnet group had a significant delay on tumor growth, which was about 10-11 days. The treatments shrank the tumor volumes for several days (day 3-day 9).

The group 8 was designed for testing the effect of WGA on the delivery system. It was clear the delivery system without WGA had much less tumor growth control effect on LLC. Irradiation+nanomag-cisplatin+magnet showed some effects at the period of treatments (day 1-day 6) but it did not last long after the treatments stopped at day 4.

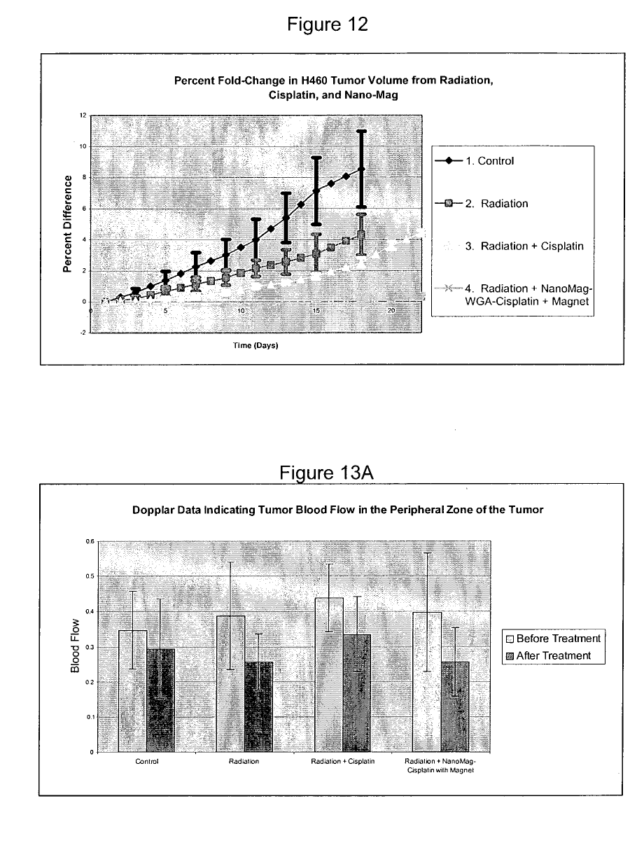

FIG. 12 shows H460 tumor volume curves after treatments with irradiation, cisplatin and nanomag-WGA-cisplatin or combinations. The radiation delayed tumor growth about 6-7 days. For the irradiation and cisplatin combination group there was an 11 days delay of tumor growth. Moreover irradiation+nanomag-WGA-Cisplatin+magnet almost totally inhibited H460 tumor growth for 20 days.

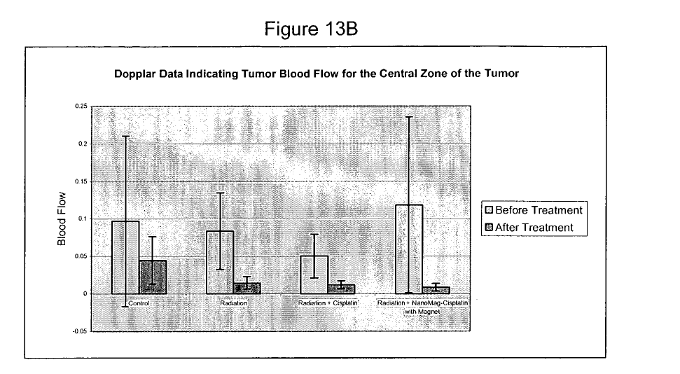

FIGS. 13A and 13B show the Doppler ultrasound data of H460 tumor model. The blood flow distribution in the peripheral zone of the tumor was reduced in all of the groups with tumor growth (control) or treatments (group 2, 3 and 4). The central blood flow supply in tumors also decreased in all of the groups, but group 4 had more significant reduction, i.e. 92.8%.

### SUMMARY OF EXAMPLES

The Examples disclosed herein show that WGA could efficiently and specifically bind to inflamed vasculature of tumor triggered by irradiation (FIGS. 7A-8B). The WGA binding on inflamed vasculature is noncontiguous and the spots are located outside of blood flow (FIG. 8A), which suggests the binding spots are not on the luminal surface of the blood vessels.

Moreover, electron microscopy data shows that most WGA binding was located at endothelial intercellular gaps. While it is not desired to be bound by a particular theory of operation, it is believed that the endothelial cells of tumors develop an acute inflammation reaction from radiation, which exposes the binding sites of WGA that are normally covered by endothelial cells and allows those sites to encounter increased blood flow because of the vasodilatation and the endothelial cells' contraction. Thus, WGA is employed as an “anchor” for the delivery vehicle, which also included iron-containing particles. In the Examples, a magnet was used to successfully pull the delivery vehicles to vasculature of tumor tissues in mouse models (FIGS. 9A-10D).

Cisplatin was chosen as the testing drug because it is presently used to enhance the effects of radiotherapy in many neoplasms. From the tumor volume studies data (FIGS. 11A-12), the delivery vehicle for cisplatin targeted the vasculature of GL-261 and H460 tumor models in mice. Tumor growth was delayed respectively 11 days and 20 days. The H460 tumor model was more sensitive than GL-261 tumor model is to the cisplatin vasculature targeting therapy. Lung cancer (H460, a human non-small cell lung cancer) are very resistant to antitumor therapies (Joseph, B., et al., Oncogene Jan. 3, 2002, 21(1):65-77; Heim, M. M., et al., J Cancer Res Clin Oncol April 2000; 126(4):198-204; Sartorius, U. A., et al., Int J Cancer Feb. 10, 2002; 97(5):584-92; Barrand, M. A., et al., Eur J Cancer 1993; 29A(3):408-15). Comparing the tumor volume change curves of group 7 with those of group 8 (FIGS. 11A-11D), irradiation+nanomag-cisplatin+magnet only (group 8) had a short period of tumor growth delay that disappeared rapidly after the treatments stopped. Since the treatment of group 8 did not include the WGA “anchor”, the nanomag-cisplatin delivery vehicle moved away with blood flow from tumor vasculature after removal of the magnet field.

The Power Doppler data (FIGS. 13A-13B) are consistent to tumor volume measurements. The greatest reduction of blood flow (92.8%) happened in the central zone of irradiation+nanomag-cisplatin+magnet group that suggest tumor necrosis was caused by the shortage of blood supply. The latter was provided via the peripheral vasculature of the tumor that was damaged by targeting therapy.

Thus, the Example disclose that the use of WGA as an “anchor” conjugated to nanomag beads can produce a relatively specific target to inflamed vasculature, can prolong the time of targeting vehicles staying in tumor, vasculature and can delay the tumor growth. The relative specificity is based on an acute inflammation reaction triggered by irradiation. The latter is an effective anti-tumor factor, which results in a collaborating therapy effect. Delivery vehicles comprising iron particles that can be pulled to tumor tissue by magnet provide an opportunity to guide the vasculature targeting vehicles to tumor. The paramagnetic delivery vehicles can have a variety of functionalized surfaces that can conjugate many chemical compounds and biological factors. Thus, the targeted delivery vehicles can be employed with different anti-tumor agents to target the vasculature of solid tumors.

### References

The references listed below as well as all references cited in the specification are incorporated herein by reference to the extent that they supplement, explain, provide a background for or teach methodology, techniques and/or compositions employed herein.

U.S. PATENTS

U.S. Pat. No. 4,695,392

U.S. Pat. No. 4,101,435

U.S. Pat. No. 6,159,443

U.S. Pat. No. 6,162,603

U.S. Pat. No. 5,843,767

U.S. Pat. No. 5,445,934

U.S. Pat. No. 5,965,352

U.S. Pat. No. 5,743,960

U.S. Pat. No. 5,916,524

U.S. Pat. No. 6,225,059

U.S. Pat. No. 6,123,819

U.S. Pat. No. 6,017,696

U.S. Pat. No. 6,245,508

U.S. Pat. No. 6,086,737

U.S. Pat. No. 5,571,388

U.S. Pat. No. 5,346,603

U.S. Pat. No. 5,534,125

U.S. Pat. No. 5,360,523

U.S. Pat. No. 5,230,781

U.S. Pat. No. 5,207,880

U.S. Pat. No. 4,729,947

U.S. Pat. No. 5,846,717

U.S. Pat. No. 5,985,557

U.S. Pat. No. 5,994,069

U.S. Pat. No. 6,001,567

U.S. Pat. No. 6,090,543

U.S. Pat. No. 6,159,443

U.S. Pat. No. 6,068,829

U.S. Pat. No. 6,232,287

U.S. Pat. No. 5,762,909

U.S. Pat. No. 6,180,084

U.S. Pat. No. 4,695,392

U.S. Pat. No. 5,248,492

U.S. Pat. No. 4,554,088

U.S. Pat. No. 5,928,944

U.S. Pat. No. 5,851,818

U.S. Pat. No. 4,235,871

U.S. Pat. No. 4,551,482

U.S. Pat. No. 6,197,333

U.S. Pat. No. 6,132,766

U.S. Pat. No. 5,011,634

U.S. Pat. No. 5,814,335

U.S. Pat. No. 6,056,938

U.S. Pat. No. 6,217,886

U.S. Pat. No. 5,948,767

U.S. Pat. No. 6,210,707

U.S. Pat. No. 5,817,636

U.S. Pat. No. 5,770,581

U.S. Pat. No. 5,641,755

U.S. Pat. No. 5,612,318

International Publications:

WO 01/23082

WO 99/32619

WO 00/66182

WO 98/10795

WO 01/09611

WO 83/03426

EP 0016552 A1

EP 0420186 A2

WO 91/09141

FR 9006-484 to Bee et al., 1990

EP 9003120 to Neveu-Prin S. et al., 1990

CA 102, P58899r, 1985

WO 00/37502

Journal Articles:

Albini A, Marchisone C, Del Grosso F, Benelli R, Masiello L, Tacchetti C, Bono M, Ferrantini M, Rozera C, Truini M, Belardelli F, Santi L & Noonan DM (2000) Inhibition of angiogenesis and vascular tumor growth by interferon- producing cells: A gene therapy approach. Am J Pathol 156:1381-1393.

Alksne J F, Fingerhut A G, Rand R W: Magnetic prob for the stereotactic thrombosis of intracranial aneurysms. J Neurol Neurosurg Psychiat 30:159-62, 1967

Alexay et al. (1996) The International Society of Optical Engineering 2705/63.

Arshady R: Microspheres for biomedical applications: Preparation of reactive and labeled microspheres. Biomaterials 14:5-15, 1993

Bacri J C, Cabuil V, Cebers C, et al: Magnetic vesicles. Mat Sci Eng C2:197-203, 1995

Bee A, Bouchami R, Brossel R, et al: Prodede d'obtention de supports magnetiques finement divises par modification controlee de la surface de particles precurseurs magnetiques chargees et produits obtenus. French Patent 1990, 9006-484, 1990

Betageri G, Jenkins S & Parsons D (1993) Liposome Drug Delivery Systems. Technomic Publishing, Lancaster.

Brush (2001) The Scientist 15(9):25-28.

Carlsson J, Drevin H, Axen R: Protein thiolation and reversible protein-protein conjugation. SPDP a new heterobifunctional reagent. Biochem J 173:723, 1978

Carpizo D & Iruela-Arispe M L (2000) Endogenous regulators of angiogenesis—emphasis on proteins with thrombospondin—type I motifs. Cancer Metastasis Rev 19:159-165.

Chan T W, Eley C, Liberti P, et al: Magnetic resonance imaging of abscesses using lipid-coated iron oxide particles. Invest Radiol 27:443-49, 1992.

Clapp C, Martial J A, Guzman R C, Rentier-Delure F & Weiner R I (1993) The 16-kilodalton N-terminal fragment of human prolactin is a potent inhibitor of angiogenesis. Endocrinology 133:1292-1299.

Cohen J I & Seidel K E (1993) Generation of varicella-zoster virus (VZV) and viral mutants from cosmid DNAs: VZV thymidylate synthetase is not essential for replication in vitro. Proc Natl Acad Sci USA 90:7376-7380.

Cruz P E, Almeida J S, Murphy P N, Moreira J L & Carrondo M J (2000b) Modeling retrovirus production for gene therapy. 1. Determination Of optimal bioreaction mode and harvest strategy. Biotechnol Prog 16:213-221.

Cruz P E, Goncalves D, Almeida J, Moreira J L & Carrondo M J (2000a) Modeling retrovirus production for gene therapy. 2. Integrated optimization of bioreaction and downstream processing. Biotechnol Prog 16:350-357.

Csermely P, Schnaider T, Soti C, Prohaszka Z & Nardai G (1998) The 90-Kda Molecular Chaperone Family: Structure, Function, and Clinical Applications. A Comprehensive Review. Pharmacol Ther 79:129-168.

Cunningham C & Davison A J (1993) A cosmid-based system for constructing mutants of herpes simplex virus type 1. Virology 197:116-124.

Dias S, Thomas H & Balkwill F (1998) Multiple molecular and cellular changes associated with tumour stasis and regression during IL-12 therapy of a murine breast cancer model. Int J Cancer 75:151-157.

Dameron K M, Volpert O V, Tainsky M A & Bouck N (1994) Control of angiogenesis in fibroblasts by p53 regulation of thrombospondin-1. Science 265:1582-1584.

de Feyter R & Li P (2000) Technology evaluation: HIV ribozyme gene therapy, Gene Shears Pty Ltd. Curr Opin Mol Ther2:332-335.

Delecluse H J & Hammerschmidt W (2000) The genetic approach to the Epstein-Barr virus: from basic virology to gene therapy. Mol Pathol 53:270-279.

DeRisi et al. (1996) Nat Genet 14:457-460

Dubiley et al. (1997) Nuc Acids Res 25:2259-2265.

Dutton A H, Tokuyasu K T, Singer J: Iron-dextran antibody conjugates: General method for simultaneous staining of two components in high resolution immunoelectron microscopy. Proc Natl Acad Sci 76:3392-96, 1979

Easton D P, Kaneko Y & Subjeck J R (2000) The HSP110 and GRP1 70 Stress Proteins: Newly Recognized Relatives of the HSP70s. Cell Stress Chaperones 5:276-290.

Ehsan A & Mann M J (2000) Antisense and gene therapy to prevent restenosis. Vasc Med 5:103-114.

Eijan A M, Davel L, Oisgold-Daga S & de Lustig E S (1991) Modulation of tumor-induced angiogenesis by proteins of extracellular matrix. Mol Biother 3:38-40.

El Bashir, Nature 411:494, 2001.

Englert (2000) in Schena, ed., Microarray Biochip Technology, pp. 231-246, Eaton Publishing, Natick, Mass., United States of America

Fabre P, Casegrande C, Veyssie M, et al: Ferrosmetics: A new magnetic and mesomorphic phase. Phys Rev Lett 64:530-33, 1990

Fahlvik A K, Holtz E, Leander P, et al: Magnetic starch microspheres: Efficacy and elimination. Invest Radiol 25:113-20, 1990

Flickinger & Trost, Eu. J. Cancer 12(2):159-60 (1976).

Fodor et al. (1991) Science 251:767-773

Fodor et al. (1993) Nature 364:555-556

Gabe, D. Radiotherapy & Oncology 30:199-205 (1994)

Goldman C K, Kendall R L, Cabrera G, Soroceanu L, Heike Y, Gillespie G Y, Siegal G P, Mao X, Bett A J, Huckle W R, Thomas K A & Curiel D T (1998) Paracrine expression of a native soluble vascular endothelial growth factor receptor inhibits tumor growth, metastasis, and mortality rate. Proc Natl Acad Sci USA 95:8795-8800.

Gossen M & Bujard H (1992) Tight Control of Gene Expression in Mammalian Cells by Tetracycline-Responsive Promoters. Proc Natl Acad Sci USA 89:5547-5551.

Gossen M, Freundlieb S, Bender G, Muller G, Hillen W & Bujard H (1995) Transcriptional Activation by Tetracyclines in Mammalian Cells. Science 268:1766-1769.

Gregoriadis G, ed (1993) Liposome Technology. CRC Press, Boca Raton, Fla.

Guha C, Chowdhury N R & Chowdhury J R (2000) Recombinant adenoassociated virus in cancer gene therapy. J. Hepatol 32:1031-1034.

Guedon et al. (2000) Anal Chem 72(24):6003-6009.

Habib N A, Hodgson H J, Lemoine N & Pignatelli M (1999) A phase I/II study of hepatic artery infusion with wtp53-CMV-Ad in metastatic malignant liver tumours. Hum Gene Ther 10:2019-2034.

Hafeli U, Schutt W, Teller J, Zborowski M (Eds), Scientific and Clinical Applications of Magnetic Carriers, Plenum Press, New York (1997).

Hainfeld, J. Proc. Natl. Acad. Sci. USA 89:11064-11068 (1992).

Halbert C L, Alexander I E, Wolgamot G M & Miller A D (1995) Adeno-associated virus vectors transduce primary cells much less efficiently than immortalized cells. J Virol 69:1473-1479.

Hallahan D E, Geng L, Cmelak A J, Chakravarthy A B, Martin W, Scarfone C & Gonzalez A (2001) Targeting drug delivery to radiation-induced neoantigens in tumor microvasculature. J Control Release 74:183-191.

Hallahan D, Clark E T, Kuchibhotla J, Gewertz B L & Collins T (1995a) E-Selectin Gene Induction by Ionizing Radiation Is Independent of Cytokine Induction. Biochem Biophys Res Commun 217:784-795.

Hauck W & Stanners C P (1995) Transcriptional Regulation of the Carcinoembryonic Antigen Gene. Identification of Regulatory Elements and Multiple Nuclear Factors. J Biol Chem 270:3602-3610.

Heaton et al. (2001) Proc Natl Acad Sci USA 98(7):3701 -3704.

Heller R, Jaroszeski M, Atkin A, Moradpour D, Gilbert R, Wands J & Nicolau C (1996) In vivo gene electroinjection and expression in rat liver. FEBS Lett 389:225-228.

Hermanson (1990) Bioconjugate Techniques, Academic Press, San Diego, Calif.

Hitt M M & Graham F L (2000) Adenovirus vectors for human gene therapy. Adv Virus Res 55:479-505.

Hofmann G A, Dev S B, Nanda G S & Rabussay D (1999) Electroporation therapy of solid tumors. Crit Rev Ther Drug Carrier Syst 16:523-569.

Ingber D, Fujita T, Kishimoto S, Sudo K, Kanamaru T, Brem H & Folkman J (1990) Synthetic analogues of fumagillin that inhibit angiogenesis and suppress tumour growth. Nature 348:555-557.

Janoff A, ed (1999) Liposomes: Rational Design. M. Dekker, New York.

Joki T, Nakamura M & Ohno T (1995) Activation of the Radiosensitive Egr-1 Promoter Induces Expression of the Herpes Simplex Virus Thymidine Kinase Gene and Sensitivity of Human Glioma Cells to Ganciclovir. Hum Gene Ther 6:1507-1513.

Kirk C J & Mule J J (2000) Gene-modified dendritic cells for use in tumor vaccines. Hum Gene Ther 11:797-806.

Komaki S & Vos J M (2000) Epstein-Barr virus vectors for gene therapy. Adv Virus Res 55:453-462.

Kosfeld M D & Frazier W A (1993) Identification of a new cell adhesion motif in two homologous peptides from the COOH-terminal cell binding domain of human thrombospondin. J Biol Chem 268:8808-8814.

Krauzewicz N & Griffin B E (2000) Polyoma and papilloma virus vectors for cancer gene therapy. Adv Exp Med Biol 465:73-82.

Kuznetsov A A, Harutyunyan A R, Dobrinsky E K, et al: Ferro-carbon particles: Preparation and chemical applications.

Kwok, C. S. et al. (1985) Med. Phys. 12:405.

Labat-Moleur F, Steffan A M, Brisson C, Perron H, Feugeas O, Furstenberger P, Oberling F, Brambilla E & Behr J P (1996) An electron microscopy study into the mechanism of gene transfer with lipopolyamines. Gene Ther 3:1010-1017.

Lasic D & Martin F, eds (1995) Stealth Liposomes. CRC Press, Boca Raton, Fla.

Latchman D S (2001) Gene delivery and gene therapy with herpes simplex virus-based vectors. Gene 264:1-9.

Leserman L D, Barbet J, Kourilsky F & Weinstein J N (1980) Targeting to cells of fluorescent liposomes covalently coupled with monoclonal antibody or protein A. Nature 288:602-604.

Lewin A S & Hauswirth W W (2001) Ribozyme gene therapy: applications for molecular medicine. Trends Mol Med 7:221-228.

Lin P, Sankar S, Shan S, Dewhirst M W, Polverini P J, Quinn T Q & Peters K G (1998a) Inhibition of tumor growth by targeting tumor endothelium using a soluble vascular endothelial growth factor receptor. Cell Growth Differ 9:49-58.

Lin P, Buxton J A, Acheson A, Radziejewski C, Maisonpierre P C, Yancopoulos G D, Channon K M, Hale L P, Dewhirst M W, George S E & Peters K G (1998b) Antiangiogenic gene therapy targeting the endothelium-specific receptor tyrosine kinase Tie2. Proc Natl Acad Sci USA 95:8829-8834.

Liu & Hlady (1996) Coll Sur B 8:25-37

Lundstrom K (1999) Alphaviruses as tools in neurobiology and gene therapy. J Recept Signal Transduct Res 19:673-686.

Mace et al. (2000) in Shena, ed., Microarray Biochip Technology, pp. 39-64, Eaton Publishing, Natick, Mass., United States of America

Maier et al. (1994) J Biotechnol 35:191-203.

Maione T E, Gray G S, Petro J, Hunt A J, Donner A L, Bauer S I, Carson H F & Sharpe R J (1990) Inhibition of angiogenesis by recombinant human platelet factor-4 and related peptides. Science 247:77-79.

Maniatis et al. (1989) Molecular Cloning: A Laboratory Manual, Cold Spring Harbor Press, Cold Spring Harbor, N.Y.

Massart R, Roger J, Cabuil V: New trends in chemistry of magnetic colloids: Polar and nonpolar magnetic fluids, emulsions, capsules and vesicles. Brazilian J Phys 25:135-141, 1995

Menager C, Babuil V: Synthesis of magnetic liposomes. J Colloid Interface Sci 169:251, 1995

Miklavcic D, Beravs K, Semrov D, Cemazar M, Demsar F & Sersa G (1998) The importance of electric field distribution for effective in vivo electroporation of tissues. Biophys J 74:2152-2158.

Molday R S, Mackenzie D: Immunospecific ferromagnetic iron-dextran reagents for the labelling and magnetic separation of cells. J Immunol Meth 52:353-67, 1982

Morishita R, Aoki M & Kaneda Y (2000) Oligonucleotide-based gene therapy for cardiovascular disease: are oligonucleotide therapeutics novel cardiovascular drugs? Curr Drug Targets 1:15-23.

Meyers P H, Cronic F, Nice C M: Experimental approach in the use and magnetic control of metallic iron particles in the lymphatic and vascular system of dogs as a contrast and isotopic agent. Am J Roentgenol 90:1068-77, 1963

Nabel G (1997) Vectors for Gene Therapy. In: Current Protocols in Human Genetics. John Wiley & Sons, New York.

Narvaiza I, Mazzolini G, Barajas M, Duarte M, Zaratiegui M, Qian C, Melero I & Prieto J (2000) Intratumoral coinjection of two adenoviruses, one encoding the chemokine IFN-gamma-inducible protein-10 and another encoding IL-12, results in marked antitumoral synergy. J Immunol 164:3112-3122.

Nelson et al. (2001) Anal Chem 73(1):1-7.

Neumann E, Schaefer-Ridder M, Wang Y & Hofschneider PH (1982) Gene transfer into mouse lyoma cells by electroporation in high electric fields. Embo J 1:841-845.

Neveu-Prin S, Cabuil V, Massart R, et al: Encapsulation of magnetic fluids. J Magn Magn Mat 122:42-45, 1993

Nomura T & Hasegawa H (2000) Chemokines and anti-cancer immunotherapy: anti-tumor effect of EBI1-ligand chemokine (ELC) and secondary lymphoid tissue chemokine (SLC). Anticancer Res 20:4073-4080.

Norris J S, Hoel B, Voeks D, Maggouta F, Dahm M, Pan W & Clawson G (2000) Design and testing of ribozymes for cancer gene therapy. Adv Exp Med Biol465:293-301.

O'Byrne K J, Dalgleish A G, Browning M J, Steward W P & Harris A L (2000) The relationship between angiogenesis and the immune response in carcinogenesis and the progression of malignant disease. Eur J Cancer 36:151-169.

O'Donnell et al. (1997) Anal Chem 69:2438-2443.

Ohtsuka K & Hata M (2000) Molecular Chaperone Function of Mammalian HSP70 and HSP40—a Review. Int J Hyperthermia 16:231-245.

O'Reilly M S, Holmgren L, Shing Y, Chen C, Rosenthal R A, Moses M, Lane W S, Cao Y, Sage E H & Folkman J (1994) Angiostatin: a novel angiogenesis inhibitor that mediates the suppression of metastases by a Lewis lung carcinoma. Cell 79:315-328.

O'Reilly M S, Boehm T, Shing Y, Fukai N, Vasios G, Lane W S, Flynn E, Birkhead J R, Olsen B R & Folkman J (1997) Endostatin: an endogenous inhibitor of angiogenesis and tumor growth. Cell 88:277-285.

Palese P, Zheng H, Engelhardt O G, Pleschka S & Garcia-Sastre A (1996) Negative-strand RNA viruses: genetic engineering and applications. Proc Natl Acad Sci USA 93:11354-11358.

Patrizio, G., et al: Cancer targeted liposomes containing superparamagnetic iron oxide: ferrosomes. Proc 8th Annual Meeting of the Society of Magnetic Resonance in Medicine (SMRM), Amsterdam, Berkeley:327, 1989.

Phillips M I, Galli S M & Mehta J L (2000) The potential role of antisense oligodeoxynucleotide therapy for cardiovascular disease. Drugs 60:239-248.

Piétu et al. (1996) Genome Res 6:492-503.

Randolph & Waggoner (1997) Nuc Acids Res 25:2923-2929.

Ratner & Castner (1997) in Vickerman, ed., Surface Analysis: The Principal Techniques.

Remington's Pharmaceutical Sciences, 16th Ed. Mack Publishing Company (1980).

Renshaw P F, Owen C S, McLaughlin A C, et al: Ferromagnetic contrast agents: A new approach. Magn Reson Med 3:217-25, 1986

Richards C A, Austin E A & Huber B E (1995) Transcriptional Regulatory Sequences of Carcinoembryonic Antigen: Identification and Use with Cytosine Deaminase for Tumor-Specific Gene Therapy. Hum Gene Ther 6:881-893.

Rigden J E, Ely J A, Macpherson J L, Gerlach W L, Sun L Q & Symonds G P (2000) The use of ribozyme gene therapy for the inhibition of HIV replication and its pathogenic sequelae. Curr Issues Mol Biol 2:61-69.

Rose (2000) in Shena, ed., Microarray Biochip Technology, pp. 19-38, Eaton Publishing, Natick, Mass., United States of America.

Rossi J J (2000) Ribozyme therapy for HIV infection. Adv Drug Deliv Rev 44:71-78.

Saini S, Stark D D, Hahn P F, et al: Ferrite particles: A superparamagnetic MR contrast agent for the reticuloendothelial system. Radiology 162:211-16, 1987

Sakamoto N, Iwahana M, Tanaka N G & Osada Y (1991) Inhibition of angiogenesis and tumor growth by a synthetic laminin peptide, CDPGYIGSR-NH2. Cancer Res 51:903-906.

Sambrook et al., eds. (1989) Molecular Cloning: A Laboratory Manual, Cold Spring Harbor Laboratory Press, Cold Spring Harbor, N.Y.

Sandig V, Hofmann C, Steinert S, Jennings G, Schlag P & Strauss M (1996) Gene transfer into hepatocytes and human liver tissue by baculovirus vectors. Hum Gene Ther 7:1937-1945.

Sapoisky & Lipshutz (1996) Genomics 33:445-456.

Sarkis C, Serguera C, Petres S, Buchet D, Ridet J L, Edelman L & Mallet J (2000) Efficient transduction of neural cells in vitro and in vivo by a baculovirus-derived vector. Proc Natl Acad Sci USA 97:14638-14643.

Schena et al. (1995) Science 270:467-470.

Schena et al. (1996) Proc Natl Acad Sci USA 93:10614-10619.

Schubiger and Edgar, Methods in Cell Biology (1994) 44:697-713.

Sersa G, Stabuc B, Cemazar M, Miklavcic D & Rudolf Z (2000) Electrochemotherapy with cisplatin: clinical experience in malignant melanoma patients. Clin Cancer Res 6:863-867.

Shalon et al. (1996) Genome Res 6:639-645.

Shaughnessy E, Lu D, Chatterjee S & Wong K K (1996) Parvoviral vectors for the gene therapy of cancer. Semin Oncol 23:159-171.

Shippy R, Lockner R, Farnsworth M & Hampel A (1999) The hairpin ribozyme. Discovery, mechanism, and development for gene therapy. Mol Biotechnol 12:117-129.

Shoemaker et al. (1996) Nat Genet 14:450-456.

Shriver-Lake (1998) in Cass & Ligler, eds., Immobilized Biomolecules in Analysis, pp. 1-14, Oxford Press, Oxford, United Kingdom

Silman N J & Fooks A R (2000) Biophysical targeting of adenovirus vectors for gene therapy. Curr Opin Mol Ther 2:524-531.

Smith R C & Walsh K (2000) Gene therapy for restenosis. Curr Cardiol Rep 2:13-23.

Smith-Arica J R & Bartlett J S (2001) Gene therapy: recombinant adeno-associated virus vectors. Curr Cardiol Rep 3:43-49.

Southern (1975) J Mol Biol 98:503-517

Srivastava A (1994) Parvovirus-based vectors for human gene therapy. Blood Cells 20:531-536.

Staba M J, Wickham T J, Kovesdi I & Hallahan D E (2000) Modifications of the fiber in adenovirus vectors increase tropism for malignant glioma models. Cancer Gene Ther 7:13-19.

Steel et al. (2000) in Schena, ed., Microarray Biochip Technology, pp. 87-118, Eaton Publishing, Natick, Mass., United States of America

Takayama K, Ueno H, Nakanishi Y, Sakamoto T, Inoue K, Shimizu K, Oohashi H & Hara N (2000) Suppression of tumor angiogenesis and growth by gene transfer of a soluble form of vascular endothelial growth factor receptor into a remote organ. Cancer Res 60:2169-2177.

Tal J (2000) Adeno-associated virus-based vectors in gene therapy. J Biomed Sci 7:279-291.

Theriault et al. (1999) in Schena, ed., DNA Microarrays: A Practical Approach, pp. 101-120, Oxford University Press Inc., New York, N.Y.

Tolsma S S, Volpert O V, Good D J, Frazier W A, Polverini P J & Bouck N (1993) Peptides derived from two separate domains of the matrix protein thrombospondin-1 have anti-angiogenic activity. J Cell Biol 122:497-511.

Turner R D, Rand R W, Bentson J R, et al: Ferromagnetic silicone necrosis of hypernephromas by selective vascular occlusion to the tumor: A new technique. J Urol 113:455-59, 1975

Ugelstad J, Kilass L, Aune O, et al: Monodisperse polymer particles.

Uhlem M, Hornes E, Olsvik O (Eds) Advances in biomagnetic separation, Eaton Publishing, Natick, Mass., 1994

Vicat J M, Boisseau S, Jourdes P, Laine M, Wion D, Bouali-Benazzouz R, Benabid A L & Berger F (2000) Muscle transfection by electroporation with high-voltage and short-pulse currents provides high-level and long-lasting gene expression. Hum Gene Ther 11:909-916.

Voest E E, Kenyon B M, O'Reilly M S, Truitt G, D'Amato R J & Folkman J (1995) Inhibition of angiogenesis in vivo by interleukin 12. J Natl Cancer Inst 87:581-586.

Vogel & Muller-Eberhard, Anal. Biochem 118(2):262-268 (1981).

Wahlfors J J, Zullo S A, Loimas S, Nelson D M & Morgan R A (2000) Evaluation of recombinant alphaviruses as vectors in gene therapy. Gene Ther 7:472-480.

Walther W & Stein U (1999) Therapeutic genes for cancer gene therapy. Mol Biotechnol 13:21-28.

Wang et al. (1998) Proc Natl Acad Sci USA 86:9717-9721.

Weichselbaum R R, Hallahan D, Fuks Z & Kufe D (1994) Radiation induction of immediate early genes: effectors of the radiation-stress response. Int J Radiat Oncol Biol Phys 30:229-234.

Wessels B. W. and R. D. Rogus (1984) Med. Phys. 11:638.

Wickham T J, Carrion M E & Kovesdi I (1995) Targeting of adenovirus penton base to new receptors through replacement of its RGD motif with other receptor-specific peptide motifs. Gene Ther 2:750-756.

Widder K J, Senyei A E, Scarppelli D G: Magnetic microspheres: a model system for site specific drug delivery in vivo. Proc Exp Biol Med 58:141-46, 1978

Worley et al. (2000) in Shena, ed., Microarray Biochip Technology, pp. 65-86, Eaton Publishing, Natick, Mass., United States of America.

Woltering E A, Barrie R, O'Dorisio T M, Arce D, Ure T, Cramer A, Holmes D, Robertson J & Fassler J (1991) Somatostatin analogues inhibit angiogenesis in the chick chorioallantoic membrane. J Surg Res 50:245-251.

Yang et al. (1998) Science 282:2244-2246 Eaton Publishing, Natick, Mass., United States of America.

Yershov et al. (1996) Proc Natl Acad Sci USA 93:4319-4918

Yeung S N & Tufaro F (2000) Replicating herpes simplex virus vectors for cancer gene therapy. Expert Opin Pharmacother 1:623-631.

Zamroe, Nature Structural Biology 8:746, 2001.

Zwiebel J A, Nagler C, Smiley J K & Cheson B D (1998) Clinical trails referral resource. Clinical trials of adenovirus-p53 gene therapy. Oncology (Huntingt) 12:1172, 1175.

It will be understood that various details of the invention may be changed without departing from the scope of the invention. Furthermore, the foregoing description is for the purpose of illustration only, and not for the purpose of limitation, as the invention is defined by the claims as set forth hereinafter.

What is claimed is:

1. A method for identifying a radiation-inducible gene, the method comprising:
(a) isolating RNA from an irradiated cell;
(b) hybridizing the isolated RNA to one or more nucleic acids from a subject; and
(c) detecting hybridization between the isolated RNA and the one or more nucleic acids to thereby identify a radiation-inducible gene.

2. The method of claim 1, wherein the irradiated cell is a cell from a cell culture or from a tissue sample.

3. The method of claim 2, wherein the tissue sample is derived from a warm-blooded vertebrate.

4. The method of claim 3, wherein the warm-blooded vertebrate is a human.

5. The method of claim 1, wherein the isolated RNA further comprises a detectable label.

6. The method of claim 1, wherein the one or more nucleic acids is selected from the group consisting of a deoxyribonucleic acid, a ribonucleic acid, and a combination thereof.

7. The method of claim 1, wherein the one or more nucleic acids each comprise a nucleotide sequence encoding a polypeptide.

8. The method of claim 1, wherein at least one of the one or more nucleic acids further comprises a detectable label.

9. The method of claim 1, wherein the one or more nucleic acids are immobilized on a solid substrate comprising a plurality of identifying positions, each of the one or more nucleic acids occupying one of the plurality of identifying positions.

10. The method of claim 9, wherein the solid substrate comprises silicon, glass, plastic, polyacrylamide, a polymer matrix, an agarose gel, a polyacrylamide gel, an organic membrane, or an inorganic membrane.

11. A method of delivering an active agent to a target tissue in a vertebrate subject, the method comprising:
(a) providing a delivery vehicle comprising an active agent and a targeting agent that binds a radiation-induced RNA molecule;
(b) exposing the target tissue to ionizing radiation; and
(c) administering a delivery vehicle to the vertebrate subject before, after, during, or combinations thereof, exposing the target tissue to the ionizing radiation, whereby the delivery vehicle localizes to a radiation-induced RNA molecule in the target tissue to thereby deliver the active agent to the target tissue.

12. The method of claim 11, wherein the targeting agent is selected from the group consisting of an antibody and a nucleic acid.

13. The method of claim 12, wherein the nucleic acid is a double-stranded RNA.

14. The method of claim 11, wherein the active agent comprises an imaging agent.

15. The method of claim 14, wherein the imaging agent is selected from the group consisting of paramagnetic, radioactive and fluorogenic ions.

16. The method of claim 15, wherein the radioactive imaging agent is selected from the group consisting of gamma-emitters, positron-emitters and x-ray-emitters.

17. The method of claim 15, wherein the radioactive imaging agent is selected from the group consisting of ⁴³K, ⁵²Fe, ⁵⁷Co, ⁶⁷Cu, ⁶⁷Ga, ⁶⁸Ga, ⁷⁷Br, ⁸¹Rb/^{81M}Kr, ^{87M}Sr, ^{99M}Tc, ¹¹¹In, ¹¹³In, ¹²³I, ¹²⁵I, ¹²⁷Cs, ¹²⁹Cs, ¹³¹I, ¹³²I, ¹⁹⁷Hg, ²⁰³Pb and ²⁰⁶Bi.

18. The method of claim 14, wherein the radioactive imaging agent is present in an amount ranging from about 0.1 to about 100 millicuries.

19. The method of claim 11, wherein the active agent comprises a therapeutic agent.

20. The method of claim 19, wherein the therapeutic agent is selected from the group consisting of a chemotherapeutic agent, a toxin, a radiotherapeutic agent, a radiosensitizing agent, a genetic construct, and combinations thereof.

21. The method of claim 20, wherein the chemotherapeutic agent is selected from the group consisting of an anti-tumor drug, a cytokine, an anti-metabolite, an alkylating agent, a hormone, methotrexate, doxorubicin, daunorubicin, cytosine arabinoside, etoposide, 5-4 fluorouracil, melphalan, chlorambucil, a nitrogen mustard, cyclophosphamide, cis-platinum, vindesine, vinca alkaloids, mitomycin, bleomycin, purothionin, macromomycin, 1,4-benzoquinone derivatives, trenimon, steroids, aminopterin, anthracyclines, demecolcine, etoposide, mithramycin, doxorubicin, daunomycin, vinblastine, neocarzinostatin, macromycin, -amanitin, and combinations thereof.

22. The method of claim 20, wherein the toxin is selected from the group consisting of Russell's Viper Venom, activated Factor IX, activated Factor X, thrombin, phospholipase C, cobra venom factor, ricin, ricin A chain, Pseudomonas exotoxin, diphtheria toxin, bovine pancreatic ribonuclease, pokeweed antiviral protein, abrin, abrin A chain, gelonin, saporin, modeccin, viscumin, volkensin and combinations thereof.

23. The method of claim 20, wherein the radiotherapeutic agent is selected from the ⁴⁷Sc, ⁶⁷Cu, ⁹⁰Y, ¹⁰⁹Pd, ¹²³I, ¹²⁵I, ¹³¹I, ¹⁸⁶Re, ¹⁸⁸Re, ¹⁹⁹Au, ²¹¹At, ²¹²Pb, ²¹²Bi, ³²P, ³³P, ⁷¹Ge, ⁷⁷As, ¹⁰³Pb, ¹⁰⁵Rh, ¹¹¹Ag, ¹¹⁹Sb, ¹²¹Sn, ¹³¹Cs, ¹⁴³Pr, ¹⁶¹Tb, ¹⁷⁷Lu, ¹⁹¹Os, ^{193M}Pt, and ¹⁹⁷Hg.

24. The method of claim 20, wherein the radiosensitizing agent is selected from the group consisting of an anti-angiogenic agent; a DNA protein kinase inhibitor; a tyrosine kinase inhibitor; a DNA repair enzyme inhibitor; nitroimidazole; metronidazole; misonidazole; a genetic construct comprising an enhancer-promoter region which is responsive to radiation, and at least one structural gene whose expression is controlled by the enhancer-promoter; boron-neutron capture reagents; and combinations thereof.

25. The method of either of claims 20 or 24, wherein the genetic construct further comprises a viral vector.

26. The method of claim 20, wherein the therapeutic agent is a chemotherapeutic agent, and the delivery vehicle comprising the chemotherapeutic agent is administered in an amount ranging from about 10 mg to about 1000 mg.

27. The method of claim 20, wherein the therapeutic agent is a toxin, and the delivery vehicle comprising the toxin is administered in an amount ranging from about 1 to about 500 μg.

28. The method of claim 20, wherein the therapeutic agent is a radiotherapeutic agent, and the delivery vehicle comprising the radiotherapeutic agent is administered in an amount ranging from about 0.5 mg to about 100 mg.

29. The method of claim 11, wherein the target tissue comprises a neoplasm.

30. The method of claim 11, wherein the vertebrate subject is a mammal.

31. The method of claim 30, wherein the mammal is a human.

32. A delivery vehicle for use in targeted delivery of an active agent, the delivery vehicle comprising a targeting agent that binds a radiation inducible RNA molecule in a target tissue.

33. The delivery vehicle of claim 32, wherein the targeting agent is selected from the group consisting of an antibody and a nucleic acid.

34. The delivery vehicle of claim 33, wherein the nucleic acid is a double-stranded RNA.

35. The delivery vehicle of claim 32, further comprising an active agent.

36. The delivery vehicle of claim 35, wherein the active agent comprises an imaging agent.

37. The delivery vehicle of claim 36, wherein the imaging agent is selected from the group consisting of paramagnetic, radioactive and fluorogenic ions.

38. The delivery vehicle of claim 37, wherein the radioactive imaging agent is selected from the group consisting of gamma-emitters, positron-emitters and x-ray-emitters.

39. The delivery vehicle of claim 37, wherein the radioactive imaging agent is selected from the group consisting of ⁴³K, ⁵²Fe, ⁵⁷Co, ⁶⁷Cu, ⁶⁷Ga, ⁶⁸Ga, ⁷⁷Br, ⁸¹Rb/^{81M}Kr, ^{87M}Sr, ^{99M}Tc, ¹¹¹In, ¹¹³In, ¹²³I, ¹²⁵I, ¹²⁷Cs, ¹²⁹Cs, ¹³¹I, ¹³²I, ¹⁹⁷Hg, ²⁰³Pb and ²⁰⁶Bi.

40. The delivery vehicle of claim 37, wherein the radioactive imaging agent is present in an amount ranging from about 0.1 to about 100 millicuries.

41. The delivery vehicle of claim 35, wherein the active agent comprises a therapeutic agent.

42. The delivery vehicle of claim 41, wherein the therapeutic agent is selected from the group consisting of a chemotherapeutic agent, a toxin, a radiotherapeutic agent, a radiosensitizing agent, a genetic construct, and combinations thereof.

43. The delivery vehicle of claim 42, wherein the chemotherapeutic agent is selected from the group consisting of an anti-tumor drug, a cytokine, an anti-metabolite, an alkylating agent, a hormone, methotrexate, doxorubicin, daunorubicin, cytosine arabinoside, etoposide, 5-4 fluorouracil, melphalan, chlorambucil, a nitrogen mustard, cyclophosphamide, cis-platinum, vindesine, vinca alkaloids, mitomycin, bleomycin, purothionin, macromomycin, 1,4-benzoquinone derivatives, trenimon, steroids, aminopterin, anthracyclines, demecolcine, etoposide, mithramycin, doxorubicin, daunomycin, vinblastine, neocarzinostatin, macromycin, -amanitin, and combinations thereof.

44. The delivery vehicle of claim 42, wherein the toxin is selected from the group consisting of Russell's Viper Venom, activated Factor IX, activated Factor X, thrombin, phospholipase C, cobra venom factor, ricin, ricin A chain, Pseudomonas exotoxin, diphtheria toxin, bovine pancreatic ribonuclease, pokeweed antiviral protein, abrin, abrin A chain, gelonin, saporin, modeccin, viscumin, volkensin and combinations thereof.

45. The delivery vehicle of claim 42, wherein the radiotherapeutic agent is selected from the group consisting of ⁴⁷Sc, ⁶⁷Cu, ⁹⁰Y, ¹⁰⁹Pd, ¹²³I, ¹²⁵I, ¹³¹I, ¹⁸⁶Re, ¹⁸⁸Re, ¹⁹⁹Au, ²¹¹At, ²¹²Pb, ²¹²Bi, ³²P, ³³P, ⁷¹Ge, ⁷⁷As, ¹⁰³Pb, ¹⁰⁵Rh, ¹¹¹Ag, ¹¹⁹Sb, ¹²¹Sn, ¹³¹Cs, ¹⁴³Pr, ¹⁶¹Tb, ¹⁷⁷Lu, ¹⁹¹Os, ^{193M}Pt, and ¹⁹⁷Hg.

46. The delivery vehicle of claim 42, wherein the radiosensitizing agent is selected from the group consisting of an anti-angiogenic agent; a DNA protein kinase inhibitor; a tyrosine kinase inhibitor; a DNA repair enzyme inhibitor; nitroimidazole; metronidazole; misonidazole; a genetic construct comprising an enhancer-promoter region which is responsive to radiation, and at least one structural gene whose expression is controlled by the enhancer-promoter; boron-neutron capture reagents; and combinations thereof.

47. The delivery vehicle of either of claims 42 or 46, wherein the genetic construct further comprises a viral vector.

48. The delivery vehicle of claim 42, wherein the therapeutic agent is a chemotherapeutic agent, and the chemotherapeutic agent is present in an amount ranging from about 10 mg to about 1000 mg.

49. The delivery vehicle of claim 42, wherein the therapeutic agent is a toxin, and the toxin is present in an amount ranging from about 1 μg to about 500 μg.

50. The delivery vehicle of claim 42, wherein the therapeutic agent is a radiotherapeutic agent, and the radiotherapeutic agent is present in an amount ranging from about 0.5 mg to about 100 mg.

51. The delivery vehicle of claim 32, further comprising a carrier.

52. The delivery vehicle of claim 51, wherein the carrier is selected from the group consisting of a nanoparticle, a microsphere, a liposome, and combinations thereof.

53. A method of dispersing a genetic construct in a target tissue, the method comprising:
(a) providing a delivery vehicle comprising a genetic construct and a paramagnetic material;
(b) administering the delivery vehicle to a target tissue; and
(c) applying a magnetic field to the target tissue to thereby disperse the genetic construct.

54. The method of claim 53, wherein the genetic construct further comprises a nucleic acid sequence encoding a polypeptide.

55. The method of claim 54, wherein the genetic construct comprises an enhancer-promoter region that is responsive to radiation, and expression of the polypeptide is controlled by the enhancer-promoter

56. The method of claim 53, wherein the genetic construct further comprises a viral vector.

57. The method of claim 53, wherein the paramagnetic material is selected from the group consisting of iron and gadolinium.

58. The method of claim 53, wherein the paramagnetic material further comprising a material that exhibits a photoelectric effect upon interaction with incident radiation.

59. The method of claim 53, wherein the paramagnetic material is in the form of a nanoparticle.

60. The method of claim 53, wherein the delivery vehicle comprises a linker that links the paramagnetic material and the genetic construct.

61. The method of claim 60, wherein the linker a peptide.

62. The method of claim 60, wherein the linker is a cleavable linker.

63. The method of claim 53, wherein the delivery vehicle further comprises a targeting agent.

64. The method of claim 63, wherein the targeting agent binds a radiation-induced RNA molecule.

65. The method of claim 53, wherein the delivery vehicle further comprises a chemotherapeutic agent, a toxin, a radiotherapeutic agent, a radiosensitizing agent, and combinations thereof.

66. The method of claim 53, wherein the administering comprises injecting the delivery vehicle into the target tissue.

67. The method of claim 53, wherein the target tissue comprises one of a cell culture and a target tissue in a subject.

68. The method of claim 67, wherein the target tissue comprises a neoplasm.

69. The method of claim 67, wherein the subject is a mammal.

70. The method of claim 69, wherein the mammal is a human.

71. A method of enhancing retention of an active agent in a target tissue in a vertebrate subject, the method comprising:
(a) providing a delivery vehicle comprising an active agent, a paramagnetic material, and a targeting agent that binds a radiation-induced target molecule;
(b) exposing the target tissue to ionizing radiation;
(c) exposing the target tissue to a magnetic field; and
(d) administering a delivery vehicle to the vertebrate subject, whereby the delivery vehicle localizes to and is retained in the target tissue.

72. The method of claim 71, wherein the active agent comprises an imaging agent.

73. The method of claim 72, wherein the imaging agent is selected from the group consisting of paramagnetic, radioactive and fluorogenic ions.

74. The method of claim 73, wherein the radioactive imaging agent is selected from the group consisting of gamma-emitters, positron-emitters and x-ray-emitters.

75. The method of claim 73, wherein the radioactive imaging agent is selected from the group consisting of ⁴³K, ⁵²Fe, ⁵⁷Co, ⁶⁷CU, ⁶⁷Ga, ⁶⁸Ga, ⁷⁷Br, ⁸¹Rb/^{81M}Kr, ^{87M}Sr, ^{99M}Tc, ¹¹¹In, ¹¹³In, ¹²³I, ¹²⁵I, ¹²⁷Cs, ¹²⁹Cs, ¹³¹I, ¹³²I, ¹⁹⁷Hg, ²⁰³Pb and ²⁰⁶Bi.

76. The method of claim 73, wherein the radioactive imaging agent is present in an amount ranging from about 0.1 to about 100 millicuries.

77. The method of claim 71, wherein the active agent comprises a therapeutic agent.

78. The method of claim 77, wherein the therapeutic agent is selected from the group consisting of a chemotherapeutic agent, a toxin, a radiotherapeutic agent, a radiosensitizing agent, a genetic construct, and combinations thereof.

79. The method of claim 78, wherein the chemotherapeutic agent is selected from the group consisting of an anti-tumor drug, a cytokine, an anti-metabolite, an alkylating agent, a hormone, methotrexate, doxorubicin, daunorubicin, cytosine arabinoside, etoposide, 5-4 fluorouracil, melphalan, chlorambucil, a nitrogen mustard, cyclophosphamide, cis-platinum, vindesine, vinca alkaloids, mitomycin, bleomycin, purothionin, macromomycin, 1,4-benzoquinone derivatives, trenimon, steroids, aminopterin, anthracyclines, demecolcine, etoposide, mithramycin, doxorubicin, daunomycin, vinblastine, neocarzinostatin, macromycin, -amanitin, and combinations thereof.

80. The method of claim 78, wherein the toxin is selected from the group consisting of Russell's Viper Venom, activated Factor IX, activated Factor X, thrombin, phospholipase C, cobra venom factor, ricin, ricin A chain, Pseudomonas exotoxin, diphtheria toxin, bovine pancreatic ribonuclease, pokeweed antiviral protein, abrin, abrin A chain, gelonin, saporin, modeccin, viscumin, volkensin and combinations thereof.

81. The method of claim 78, wherein the radiotherapeutic agent is selected from the ⁴⁷Sc, ⁶⁷Cu, ⁹⁰Y, ¹⁰⁹Pd, ¹²³I, ¹²⁵I, ¹³¹I, ¹⁸⁶Re, ¹⁸⁸Re, ¹⁹⁹Au, ²¹¹At, ²¹²Pb, ²¹²Bi, ³²P, ³³P, ⁷¹Ge, ⁷⁷As, ¹⁰³Pb, ¹⁰⁵Rh, ¹¹¹Ag, ¹¹⁹Sb, ¹²¹Sn, ¹³¹Cs, ¹⁴³Pr, ¹⁶¹Tb, ¹⁷⁷Lu, ¹⁹¹Os, ^{193M}Pt, and ¹⁹⁷Hg.

82. The method of claim 78, wherein the radiosensitizing agent is selected from the group consisting of an anti-angiogenic agent; a DNA protein kinase inhibitor; a tyrosine kinase inhibitor; a DNA repair enzyme inhibitor; nitroimidazole; metronidazole; misonidazole; a genetic construct comprising an enhancer-promoter region which is responsive to radiation, and at least one structural gene whose expression is controlled by the enhancer-promoter; boron-neutron capture reagents; and combinations thereof.

83. The method of either of claims 78 or 82, wherein the genetic construct further comprises a viral vector.

84. The method of claim 78, wherein the therapeutic agent is a chemotherapeutic agent, and the delivery vehicle comprising the chemotherapeutic agent is administered in an amount ranging from about 10 mg to about 1000 mg.

85. The method of claim 78, wherein the therapeutic agent is a toxin, and the delivery vehicle comprising the toxin is administered in an amount ranging from about 1 to about 500 μg.

86. The method of claim 78, wherein the therapeutic agent is a radiotherapeutic agent, and the delivery vehicle comprising the radiotherapeutic agent is administered in an amount ranging from about 0.5 mg to about 100 mg.

87. The method of claim 71, wherein the paramagnetic material is selected from the group consisting of iron and gadolinium.

88. The method of claim 71, wherein the paramagnetic material is in the form of a nanoparticle.

89. The method of claim 71, wherein the delivery vehicle comprises a linker that links the paramagnetic material and the active agent.

90. The method of claim 89, wherein the linker a peptide.

91. The method of claim 89, wherein the linker is a cleavable linker.

92. The method of claim 71, wherein the target tissue comprises a neoplasm.

93. The method of claim 71, wherein the vertebrate subject is a mammal.

94. The method of claim 93, wherein the mammal is a human.
